(19) United States
(12) Patent Application Publication
(10) Pub. No.: US 2026/0137508 A1
(43) Pub. Date: May 21, 2026

(54) PROSTHETIC VALVE DOCKETING DEVICE

(71) Applicant: Edwards Lifesciences Corporation, Irvine, CA (US)

(72) Inventors: Tram Ngoc Nguyen, Santa Ana, CA (US); Jocelyn Chau, Buena Park, CA (US)

(21) Appl. No.: 19/450,474

(22) Filed: Jan. 15, 2026

Related U.S. Application Data
(63) Continuation of application No. PCT/US2024/037026, filed on Jul. 8, 2024.
(60) Provisional application No. 63/514,553, filed on Jul. 19, 2023.

(51) Int. Cl. A61F 2/24

(52) U.S. Cl. CPC A61F 2/2412; A61F 2/246; A61F 2210/0014; A61F 2220/0008; A61F 2230/0004; A61F 2230/0045; A61F 2230/0091; A61F 2240/001; A61F 2250/0037; A61F 2250/0069

(57) ABSTRACT

A docking device for securing a prosthetic valve at a native valve includes a coil having a plurality of helical turns when deployed at the native valve, and a guard member attached to the coil. The guard member is movable between a radially compressed state and a radially expanded state. The guard member includes a plurality of anchor members and a plurality of expandable members. The anchor members are not radially movable relative to the coil when the guard member moves from the radially compressed state to the radially expanded state. The expandable members project radially outwardly from the coil when the guard member moves from the radially compressed state to the radially expanded state.

### CROSS-REFERENCE TO RELATED APPLICATIONS

This application is a continuation of PCT Patent Application No. PCT/US 2024/037026 filed on Jul. 8, 2024, which claims the benefit of U.S. Provisional Application No. 63/514,553 filed Jul. 19, 2023, each of these applications being incorporated by reference herein in its entirety.

### FIELD

The present disclosure concerns examples of a docking device configured to secure a prosthetic valve at a native heart valve, as well as methods of assembling such devices.

### BACKGROUND

Prosthetic valves can be used to treat cardiac valvular disorders. Native heart valves (for example, the aortic, pulmonary, tricuspid and mitral valves) function to prevent backward flow or regurgitation, while allowing forward flow. These heart valves can be rendered less effective by congenital, inflammatory, infectious conditions, etc. Such conditions can eventually lead to serious cardiovascular compromise or death. For many years, the doctors attempted to treat such disorders with surgical repair or replacement of the valve during open heart surgery.

A transcatheter technique for introducing and implanting a prosthetic heart valve using a catheter in a manner that is less invasive than open heart surgery can reduce complications associated with open heart surgery. In this technique, a prosthetic valve can be mounted in a compressed state on the end portion of a catheter and advanced through a blood vessel of the patient until the valve reaches the implantation site. The valve at the catheter tip can then be expanded to its functional size at the site of the defective native valve, such as by inflating a balloon on which the valve is mounted or, for example, the valve can have a resilient, self-expanding frame that expands the valve to its functional size when it is advanced from a delivery sheath at the distal end of the catheter. Optionally, the valve can have a balloon-expandable, self-expanding, mechanically expandable frame, and/or a frame expandable in multiple or a combination of ways.

In some instances, a transcatheter heart valve (THV) may be appropriately sized to be placed inside a particular native valve (for example, a native aortic valve). As such, the THV may not be suitable for implantation at another native valve (for example, a native mitral valve) and/or in a patient with a larger native valve. Additionally, or alternatively, the native tissue at the implantation site may not provide sufficient structure for the THV to be secured in place relative to the native tissue. Accordingly, improvements to THVs and the associated transcatheter delivery apparatus are desirable.

### SUMMARY

The present disclosure relates to methods and devices for treating valvular regurgitation and/or other valve issues. Specifically, the present disclosure is directed to a docking device configured to receive a prosthetic valve and the methods of assembling the docking device and implanting the docking device.

A docking device for securing a prosthetic valve at a native valve can include a coil comprising a plurality of helical turns when deployed at the native valve. In addition to these features, a docking device can further comprise one or more of the components disclosed herein.

In some examples, a docking device can include a guard member attached to the coil and is movable between a radially compressed state and a radially expanded state.

In some examples, the guard member includes a plurality of anchor members and a plurality of expandable members.

In some examples, the anchor members are not radially movable relative to the coil when the guard member moves from the radially compressed state to the radially expanded state.

In some examples, the expandable members project radially outwardly from the coil when the guard member moves from the radially compressed state to the radially expanded state.

In some examples, the plurality of anchor members and the plurality of expandable members are interconnected to form a unitary piece.

In some examples, the guard member includes a plurality of discrete expandable units, each expandable unit including one anchor member and two or more expandable members fixedly connected to the anchor member.

In some examples, the guard member is not axially movable relative to the coil.

In some examples, the guard member includes a shape memory alloy.

In some examples, the docking device includes an outer cover surrounding an outer surface of the guard member.

In some examples, the docking device includes an inner cover fixedly attached to an outer surface of the coil, and the plurality of anchor members directly contact the inner cover.

In some examples, the plurality of anchor members are fixedly attached to the coil and the plurality of expandable members can cantilever over respective anchor members.

Certain aspects of the disclosure concern a method of making a docking device for securing a prosthetic valve at a native valve. The method includes receiving a tube comprising a shape memory material, transforming the tube into a cylindrical mesh structure comprising a plurality of anchor members and a plurality of expandable members that are interconnected to form a unitary piece, and shape setting the plurality of expandable members so that the plurality of expandable members cantilever over respective anchor members and project radially outwardly when no external force is applied to the plurality of expandable members.

Certain aspects of the disclosure concern another method of making a docking device for securing a prosthetic valve at a native valve. The method includes receiving a plurality of discrete expandable units, each expandable unit including one tubular member and two or more expandable members that are fixedly connected to the tubular member; and sliding the plurality of expandable units over a portion of a coil so that the plurality of expandable units are stacked on one another. The coil is configured to move from a substantially straight configuration to a helical configuration when deployed at the native valve.

Certain aspects of the disclosure concern a method for implanting a prosthetic valve. The method includes deploying a docking device at an annulus of a native valve, and deploying a prosthetic valve within the docking device. The docking device can be any of the docking devices described herein.

The above method can be performed on a living animal or on a simulation, such as on a cadaver, cadaver heart, anthropomorphic ghost, simulator (for example, with body parts, heart, tissue, etc. being simulated).

In some examples, a docking device or a guard member comprises one or more of the components recited in Examples 1-48 described in the section “Additional Examples of the Disclosed Technology” below.

The foregoing and other objects, features, and advantages of the disclosed technology will become more apparent from the following detailed description, which proceeds with reference to the accompanying figures.

### BRIEF DESCRIPTION OF THE DRAWINGS

FIG. 1 schematically illustrates a first stage in an exemplary mitral valve replacement procedure where a guide catheter and a guidewire are inserted into a vasculature of a patient and navigated through the vasculature and into a heart of the patient, towards a native mitral valve of the heart.

FIG. 2A schematically illustrates a second stage in the exemplary mitral valve replacement procedure where a docking device delivery apparatus extending through the guide catheter is used to deploy a docking device at the native mitral valve.

FIG. 2B schematically illustrates a third stage in the exemplary mitral valve replacement procedure where the docking device of FIG. 2A is fully implanted at the native mitral valve of the patient and the docking device delivery apparatus has been removed from the patient.

FIG. 3A schematically illustrates a fourth stage in the exemplary mitral valve replacement procedure where a prosthetic heart valve delivery apparatus extending through the guide catheter is deploy a prosthetic heart valve within the implanted docking device at the native mitral valve.

FIG. 3B schematically illustrates a fifth stage in the exemplary mitral valve replacement procedure where the prosthetic heart valve is fully implanted within the docking device at the native mitral valve and the prosthetic heart valve delivery apparatus has been removed from the patient.

FIG. 4 schematically illustrates a sixth stage in the exemplary mitral valve replacement procedure where the guide catheter and the guidewire have been removed from the patient.

FIG. 5 is a side perspective view of a docking device in a deployed configuration, the docking device including a helical coil and a guard member, according to one example.

FIG. 6A is a side perspective view of a portion of a guard member in a radially compressed state, according to one example.

FIG. 6B is a flattened view of the portion of the guard member of FIG. 6A.

FIG. 7A depicts an anchor member of the guard member of FIG. 6A.

FIG. 7B depicts an expandable member of the guard member of FIG. 6A.

FIG. 7C is a flattened view of a circumferential row of anchor members and expandable members of the guard member of FIG. 6A.

FIG. 7D is flattened view depicting interconnection of three anchor members of the guard member of FIG. 6A.

FIG. 8A is a side view of a segment of a docking device having the guard member of FIG. 6A in a radially compressed state, according to one example.

FIG. 8B is a side view of the segment of the docking device of FIG. 8A with the guard member in a radially expanded state, according to one example.

FIG. 8C is a side view of the segment of the docking device of FIG. 8A with the guard member in a radially expanded state, according to another example.

FIG. 8D is a cross-sectional view of the docking device of FIG. 8A taken along the line 8D-8D depicted in FIG. 8A.

FIG. 9A schematically illustrates a portion of a guard member comprising multiple stacked expandable units in a radially expanded state, according to one example.

FIG. 9B schematically illustrates the portion of the guard member of FIG. 9A, wherein the expandable units are in a radially compressed state.

FIG. 9C schematically illustrates sliding two expandable units of FIG. 9A over a portion of a coil, according to one example.

FIG. 10A schematically depicts a side view of one of the expandable units of FIG. 9A, according to one example.

FIG. 10B schematically depicts a flattened view of the expandable unit of FIG. 10A.

FIG. 10C schematically depicts a top view of the expandable unit of FIG. 10A.

FIG. 11 depicts a docking device being deployed at a native heart valve annulus, according to one example.

FIG. 12 is a perspective view of an example prosthetic heart valve.

### DETAILED DESCRIPTION

### General Considerations

It should be understood that the disclosed examples can be adapted to deliver and implant prosthetic devices in any of the native annuluses of the heart (for example, the pulmonary, mitral, and tricuspid annuluses), and can be used with any of various delivery approaches (for example, retrograde, antegrade, transseptal, transventricular, transatrial, etc.).

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the terms “coupled” and “connected” generally mean electrically, electromagnetically, and/or physically (for example, mechanically or chemically) coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient's body), while distal motion of the device is motion of the device away from the user and toward the implantation site (for example, into the patient's body). The terms “longitudinal” and “axial” refer to an axis extending in the proximal and distal directions, unless otherwise expressly defined.

Directions and other relative references (for example, inner, outer, upper, lower, etc.) may be used to facilitate discussion of the drawings and principles herein, but are not intended to be limiting. For example, certain terms may be used such as “inside,” “outside,”, “top,” “down,” “interior,” “exterior,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same. As used herein, “and/or” means “and” or “or,” as well as “and” and “or.”

### Exemplary Transcatheter Heart Valve Replacement Procedure

Described herein are various systems, apparatuses, methods, or the like, that can be used in or with delivery apparatuses to deliver a prosthetic implant (for example, a prosthetic valve, a docking device, etc.) into a patient body.

In certain examples, a delivery apparatus can be configured to deliver and implant a docking device at an implantation site, such as a native valve annulus. The docking device can be configured to more securely hold an expandable prosthetic valve implanted within the docking device, at the native valve annulus. For example, a docking device can provide or form a more circular and/or stable anchoring site, landing zone, or implantation zone at the implant site, in which a prosthetic valve can be expanded or otherwise implanted. By providing such anchoring or docking devices, replacement prosthetic valves can be more securely implanted and held at various valve annuluses, including at the mitral annulus which does not have a naturally circular cross-section.

In some examples, the docking device can be arranged within an outer shaft of the delivery apparatus. A sleeve shaft can cover or surround the docking device within the delivery apparatus and during delivery to a target implantation site. A pusher shaft can be disposed within the outer shaft, proximal to the docking device, and configured to push the docking device out of the outer shaft to position the docking device at the target implantation site. The sleeve shaft can also surround the pusher shaft within the outer shaft of the delivery apparatus. After positioning the docking device at the target implantation site, the sleeve shaft can be removed from the docking device and retracted back into the outer shaft of the delivery apparatus.

Fluid (for example, a flush fluid, such as heparinized saline or the like) can be provided to a pusher shaft lumen defined within an interior of the pusher shaft, a delivery shaft lumen defined between the sleeve shaft and the outer shaft of the delivery apparatus, and a sleeve shaft lumen defined between the pusher shaft and the sleeve shaft. By providing a consistent flow of fluid through these lumens of the delivery apparatus, stagnation of blood within the delivery apparatus can be reduced or avoided, thereby reducing a risk of thrombus formation.

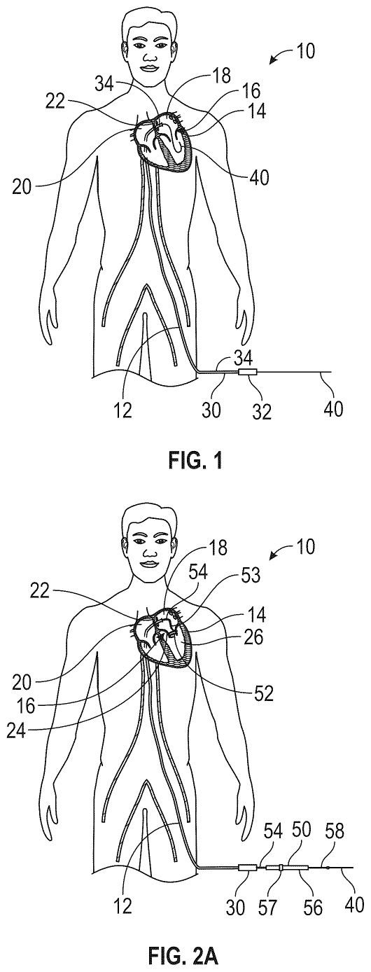

An exemplary transcatheter heart valve replacement procedure which utilizes a first delivery apparatus to deliver a docking device to a native valve annulus and then a second delivery apparatus to deliver a prosthetic heart valve (for example, THV) inside the docking device is depicted in the schematic illustrations of FIGS. 1-4.

As introduced above, defective native heart valves may be replaced with THVs. However, in certain instances, such THVs may not be able to sufficiently secure themselves to the native tissue (for example, to the leaflets and/or annulus of the native heart valve) and may undesirably shift around relative to the native tissue, leading to paravalvular leakage, valve malfunction, and/or other issues. Thus, a docking device may be implanted first at the native valve annulus and then the THV can be implanted within the docking device to help anchor the THV to the native tissue and provide a seal between the native tissue and the THV.

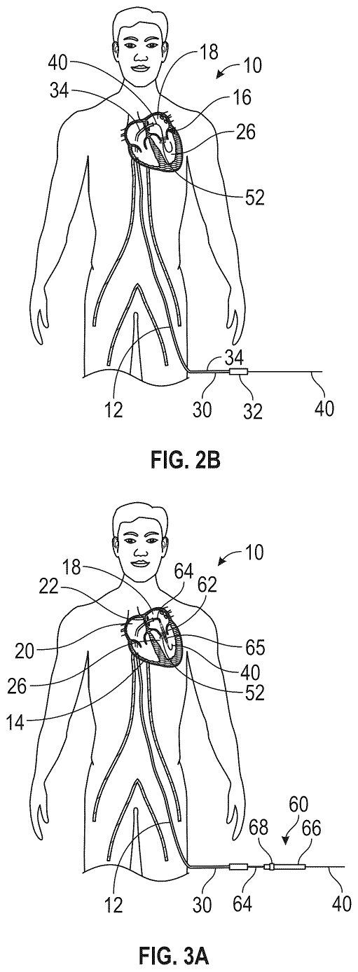

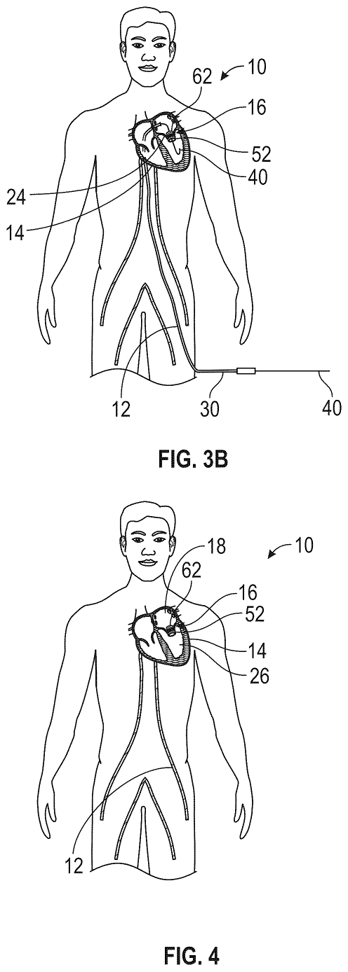

FIGS. 1-4 depict an exemplary transcatheter heart valve replacement procedure (for example, a mitral valve replacement procedure) which utilizes a docking device 52 and a prosthetic heart valve 62, according to one example. During the procedure, a user can create a pathway to a patient's native heart valve using a guide catheter 30 (FIG. 1). The user can deliver and implant the docking device 52 at the patient's native heart valve using a docking device delivery apparatus 50 (FIG. 2A) and then removes the docking device delivery apparatus 50 from the patient 10 after implanting the docking device 52 (FIG. 2B). The user can then implant the prosthetic heart valve 62 within the implanted docking device 52 using a prosthetic valve delivery apparatus 60 (FIG. 3A). Thereafter, the user can remove the prosthetic valve delivery apparatus 60 from the patient 10 (FIG. 3B), as well as the guide catheter 30 (FIG. 4).

FIG. 1 depicts a first stage in a mitral valve replacement procedure, according to one example. As shown, the guide catheter 30 and a guidewire 40 can be inserted into a vasculature 12 of a patient 10 and navigated through the vasculature 12, into a heart 14 of the patient 10, and toward the native mitral valve 16. Together, the guide catheter 30 and the guidewire 40 can provide a path for the docking device delivery apparatus 50 and the prosthetic valve delivery apparatus 60 to be navigated through and along, to the implantation site (for example, the native mitral valve 16 or native mitral valve annulus).

Initially, the user may first make an incision in the patient's body to access the vasculature 12. For example, as illustrated in FIG. 1, the user may make an incision in the patient's groin to access a femoral vein. Thus, in such examples, the vasculature 12 may include a femoral vein.

After making the incision to access the vasculature 12, the user may insert the guide catheter 30, the guidewire 40, and/or additional devices (such as an introducer device or transseptal puncture device) through the incision and into the vasculature 12. The guide catheter 30 (which can also be referred to as an “introducer device,” “introducer,” or “guide sheath”) can be configured to facilitate the percutaneous introduction of various implant delivery devices (for example, the docking device delivery apparatus 50 and the prosthetic valve delivery apparatus 60) into and through the vasculature 12 and may extend through the vasculature 12 and into the heart 14 but may stop short of the native mitral valve 16. The guide catheter 30 can comprise a handle 32 and a shaft 34 extending distally from the handle 32. The shaft 34 can extend through the vasculature 12 and into the heart 14 while the handle 32 can remain outside the body of the patient 10 and can be operated by the user in order to manipulate the shaft 34 (FIG. 1).

The guidewire 40 can be configured to guide the delivery apparatuses (for example, the guide catheter 30, the docking device delivery apparatus 50, the prosthetic valve delivery apparatus 60, additional catheters, or the like) and their associated devices (for example, docking device, prosthetic heart valve, and the like) to the implantation site within the heart 14, and thus may extend all the way through the vasculature 12 and into a left atrium 18 of the heart 14 (and in some examples, through the native mitral valve 16 and into a left ventricle of the heart 14) (FIG. 1).

In some instances, a transseptal puncture device or catheter can be used to initially access the left atrium 18, prior to inserting the guidewire 40 and the guide catheter 30. For example, after making the incision to access the vasculature 12, the user may insert a transseptal puncture device through the incision and into the vasculature 12. The user may guide the transseptal puncture device through the vasculature 12 and into the heart 14 (for example, through the femoral vein and into the right atrium 20). The user can then make a small incision in an atrial septum 22 of the heart 14 to allow access to the left atrium 18 from the right atrium 20. The user can then insert and advance the guidewire 40 through the transseptal puncture device within the vasculature 12 and through the incision in the atrial septum 22 into the left atrium 18. Once the guidewire 40 is positioned within the left atrium 18 and/or the left ventricle 26, the transseptal puncture device can be removed from the patient 10. The user can then insert the guide catheter 30 into the vasculature 12 and advance the guide catheter 30 into the left atrium 18 over the guidewire 40 (FIG. 1).

In some instances, an introducer device can be inserted through a lumen of the guide catheter 30 prior to inserting the guide catheter 30 into the vasculature 12. In some instances, the introducer device can include a tapered end that extends out a distal tip of the guide catheter 30 and that is configured to guide the guide catheter 30 into the left atrium 18 over the guidewire 40. Additionally, in some instances the introducer device can include a proximal end portion that extends out a proximal end of the guide catheter 30. Once the guide catheter 30 reaches the left atrium 18, the user can remove the introducer device from inside the guide catheter 30 and the patient 10. Thus, only the guide catheter 30 and the guidewire 40 remain inside the patient 10. The guide catheter 30 is then in position to receive an implant delivery apparatus and help guide it to the left atrium 18, as described further below.

FIG. 2A depicts a second stage in the exemplary mitral valve replacement procedure where a docking device 52 can be implanted at the native mitral valve 16 of the heart 14 of the patient 10 using a docking device delivery apparatus 50 (which may also be referred to as an “implant catheter,” or a “docking device delivery device,” or simply “delivery apparatus”).

In general, the docking device delivery apparatus 50 can include a delivery shaft 54 (which may also be referred to as an “outer shaft”), a handle 56, and a pusher assembly 58 (which may also be referred to as a “pusher shaft”). The delivery shaft 54 can be configured to be advanced through the patient's vasculature 12 and to the implantation site (for example, native mitral valve 16) by the user, and may be configured to retain the docking device 52 in a distal end portion 53 of the delivery shaft 54. In some examples, the distal end portion 53 of the delivery shaft 54 can retain the docking device 52 therein in a substantially straightened delivery configuration.

The handle 56 of the docking device delivery apparatus 50 can be configured to be gripped and/or otherwise held by the user to advance the delivery shaft 54 through the patient's vasculature 12. Specifically, the handle 56 can be coupled to a proximal end of the delivery shaft 54 and can be configured to remain accessible to the user (for example, outside the body of the patient 10) during the docking device implantation procedure. In this way, the user can advance the delivery shaft 54 through the patient's vasculature 12 by exerting a force on (for example, pushing) the handle 56. In some examples, the delivery shaft 54 can be configured to carry the pusher assembly 58 and/or the docking device 52 with it as it advances through the patient's vasculature 12. In this way, the docking device 52 and/or the pusher assembly 58 can advance through the patient's vasculature 12 in lockstep with the delivery shaft 54 as the user grips the handle 56 and pushes the delivery shaft 54 deeper into the patient's vasculature 12.

In some examples, the handle 56 can comprise one or more articulation members 57 that are configured to aid in navigating the delivery shaft 54 through the vasculature 12. For example, the one or more articulation members 57 can comprise one or more of knobs, buttons, wheels, and/or other types of physically adjustable control members that are configured to be adjusted by the user to flex, bend, twist, turn, and/or otherwise articulate a distal end portion 53 of the delivery shaft 54 to aid in navigating the delivery shaft 54 through the vasculature 12 and/or within the heart 14.

The pusher assembly 58 can be configured to deploy and/or implant the docking device 52 at the implantation site (for example, the native mitral valve 16). For example, the pusher assembly 58 can be configured to be adjusted by the user to push the docking device 52 out of the distal end portion 53 of the delivery shaft 54. A pusher shaft of the pusher assembly 58 can extend through the delivery shaft 54 and can be disposed adjacent to the docking device 52 within the delivery shaft 54. In some examples, the docking device 52 can be releasably coupled to the pusher shaft of the pusher assembly 58 via a connection mechanism of the docking device delivery apparatus 50 such that the docking device 52 can be released after being deployed at the native mitral valve 16. Because the docking device 52 is retained by, held, and/or otherwise coupled to the pusher assembly 58, the docking device 52 can advance in lockstep with the pusher assembly 58 through and/or out of the delivery shaft 54.

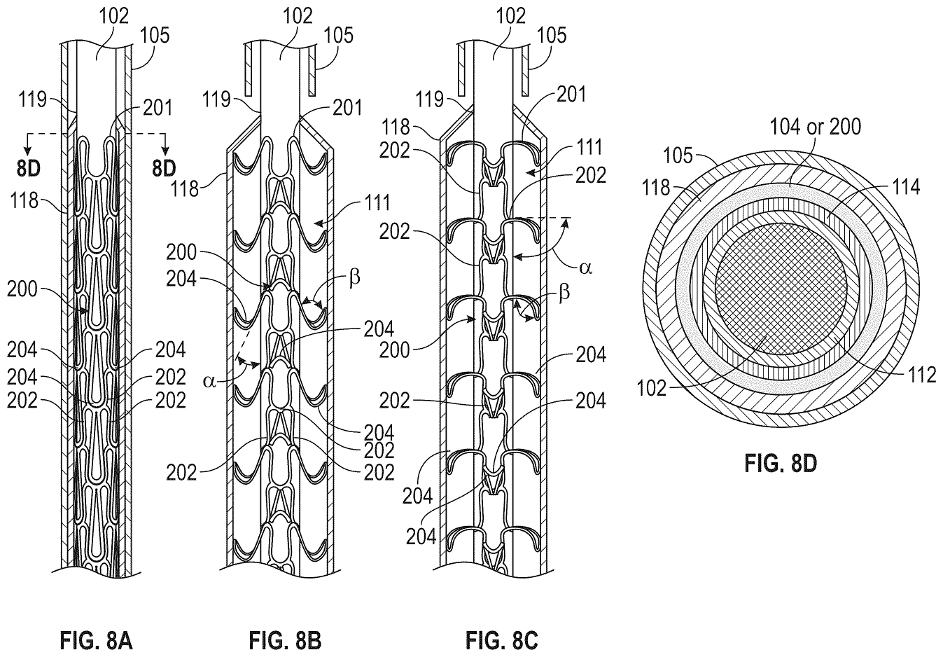

In addition to the pusher shaft, in certain instances, the pusher assembly 58 can also include a sleeve shaft. The pusher shaft can be configured to advance the docking device 52 through the delivery shaft 54 and out of the distal end portion 53 of the delivery shaft 54, while the sleeve shaft, when included, can have a distal dock sleeve (see, for example, dock sleeve 105 depicted in FIGS. 8A-8D) configured to cover the docking device 52 within the delivery shaft 54 and while pushing the docking device 52 out of the delivery shaft 54 and positioning the docking device 52 at the implantation site. In some examples, the pusher shaft can be covered, at least in part, by the sleeve shaft.

In some examples, the pusher assembly 58 can comprise a pusher handle that is coupled to the pusher shaft and that is configured to be gripped and pushed by the user to translate the pusher shaft axially relative to the delivery shaft 54 (for example, to push the pusher shaft into and/or out of the distal end portion 53 of the delivery shaft 54). The dock sleeve can be configured to be retracted and/or withdrawn from the docking device 52, after positioning the docking device 52 at the target implantation site. For example, the pusher assembly 58 can include a sleeve handle that is coupled to the sleeve shaft and is configured to be pulled by a user to retract (for example, axially move) the sleeve shaft relative to the pusher shaft, thereby retracting the dock sleeve.

The pusher assembly 58 can be removably coupled to the docking device 52, and as such can be configured to release, detach, decouple, and/or otherwise disconnect from the docking device 52 once the docking device 52 has been deployed at the target implantation site. As just one example, the pusher assembly 58 may be removably coupled to the docking device 52 via a thread, string, yarn, suture, or other suitable material that is tied or sutured to the docking device 52.

In some examples, the pusher assembly 58 can include a suture lock assembly (also referred to as a “suture lock”) that is configured to receive and/or hold the thread or other suitable material that is coupled to the docking device 52 via a suture. The thread or other suitable material that forms the suture can extend from the docking device 52, through the pusher assembly 58, to the suture lock assembly. The suture lock assembly can also be configured to cut the suture to release, detach, decouple, and/or otherwise disconnect the docking device 52 from the pusher assembly 58. For example, the suture lock assembly can comprise a cutting mechanism that is configured to be adjusted by the user to cut the suture.

Referring again to FIG. 2A, after the guide catheter 30 is positioned within the left atrium 18, the user may insert the docking device delivery apparatus 50 (for example, the delivery shaft 54) into the patient 10 by advancing the delivery shaft 54 of the docking device delivery apparatus 50 through the guide catheter 30 and over the guidewire 40. In some examples, the guidewire 40 can be at least partially retracted away from the left atrium 18 and into the guide catheter 30. The user may then continue to advance the delivery shaft 54 of the docking device delivery apparatus 50 through the vasculature 12 along the guidewire 40 until the delivery shaft 54 reaches the left atrium 18, as illustrated in FIG. 2A. Specifically, the user may advance the delivery shaft 54 of the docking device delivery apparatus 50 by gripping and exerting a force on (for example, pushing) the handle 56 of the docking device delivery apparatus 50 toward the patient 10. While advancing the delivery shaft 54 through the vasculature 12 and the heart 14, the user may adjust the one or more articulation members 57 of the handle 56 to navigate the various turns, corners, constrictions, and/or other obstacles in the vasculature 12 and the heart 14.

Once the delivery shaft 54 reaches the left atrium 18 and extends out of a distal end of the guide catheter 30, the user can position the distal end portion 53 of the delivery shaft 54 at and/or near the posteromedial commissure of the native mitral valve 16 using the handle 56 (for example, the articulation members 57). The user may then push the docking device 52 out of the distal end portion 53 of the delivery shaft 54 with the shaft of the pusher assembly 58 to deploy and/or implant the docking device 52 within the annulus of the native mitral valve 16.

In some examples, the docking device 52 may be constructed from, formed of, and/or comprise a shape memory material, and as such, may return to its original, pre-formed shape when it exits the delivery shaft 54 and is no longer constrained by the delivery shaft 54. As one example, the docking device 52 may originally be formed as a coil, and thus may wrap around leaflets 24 of the native mitral valve 16 as it exits the delivery shaft 54 and returns to its original coiled configuration.

After pushing a ventricular portion of the docking device 52 (for example, the portion of the docking device 52 shown in FIG. 2A that is configured to be positioned within a left ventricle 26 and/or on the ventricular side of the native mitral valve 16), the user may then deploy the remaining portion of the docking device 52 (for example, an atrial portion of the docking device 52) from the delivery shaft 54 within the left atrium 18 by retracting the delivery shaft 54 away from the medial commissure of the native mitral valve 16. For example, the user can maintain the position of the pusher assembly 58 (for example, by exerting a holding and/or pushing force on the pusher shaft) while retracting the delivery shaft 54 proximally so that the delivery shaft 54 withdraws and/or otherwise retracts relative to the docking device 52 and the pusher assembly 58. In this way, the pusher assembly 58 can hold the docking device 52 in place while the user retracts the delivery shaft 54, thereby releasing the docking device 52 from the delivery shaft 54. In some examples, the user can also remove the dock sleeve from the docking device 52, for example, by retracting the sleeve shaft.

After deploying and implanting the docking device 52 at the native mitral valve 16, the user may disconnect the docking device delivery apparatus 50 from the docking device 52. Once the docking device 52 can be disconnected from the docking device delivery apparatus 50 (for example, by cutting the suture tied to the docking device 52), the user may retract the docking device delivery apparatus 50 out of the vasculature 12 and away from the patient 10 so that the user can deliver and implant a prosthetic heart valve 62 within the implanted docking device 52 at the native mitral valve 16.

FIG. 2B depicts a third stage in the mitral valve replacement procedure, where the docking device 52 has been fully deployed and implanted at the native mitral valve 16 and the docking device delivery apparatus 50 (including the delivery shaft 54) has been removed from the patient 10 such that only the guidewire 40 and the guide catheter 30 remain inside the patient 10. In some examples, after removing the docking device delivery apparatus, the guidewire 40 can be advanced out of the guide catheter 30, through the implanted docking device 52 at the native mitral valve 16, and into the left ventricle 26 (FIG. 2A). As such, the guidewire 40 can help to guide the prosthetic valve delivery apparatus 60 through the annulus of the native mitral valve 16 and at least partially into the left ventricle 26.

As illustrated in FIG. 2B, the docking device 52 can comprise a plurality of helical turns that wrap around the leaflets 24 of the native mitral valve 16 (within the left ventricle 26). The implanted docking device 52 can have a more cylindrical shape than the annulus of the native mitral valve 16, thereby providing a geometry that more closely matches the shape or profile of the prosthetic heart valve to be implanted. As a result, the docking device 52 can provide a tighter fit, and thus a better seal, between the prosthetic heart valve and the native mitral valve 16, as described further below.

FIG. 3A depicts a fourth stage in the mitral valve replacement procedure where the user is delivering and/or implanting a prosthetic heart valve 62 within the docking device 52 using a prosthetic valve delivery apparatus 60.

As shown in FIG. 3A, the prosthetic valve delivery apparatus 60 can comprise a delivery shaft 64 and a handle 66. The delivery shaft 64 can extend distally from the handle 66. The delivery shaft 64 can be configured to extend into the patient's vasculature 12 to deliver, implant, expand, and/or otherwise deploy the prosthetic heart valve 62 within the docking device 52 at the native mitral valve 16. The handle 66 can be configured to be gripped and/or otherwise held by the user to advance the delivery shaft 64 through the patient's vasculature 12.

In some examples, the handle 66 can comprise one or more articulation members 68 that are configured to aid in navigating the delivery shaft 64 through the vasculature 12 and the heart 14. Specifically, the articulation members 68 can comprise one or more of knobs, buttons, wheels, and/or other types of physically adjustable control members that are configured to be adjusted by the user to flex, bend, twist, turn, and/or otherwise articulate a distal end portion of the delivery shaft 64 to aid in navigating the delivery shaft 64 through the vasculature 12 and into the left atrium 18 and left ventricle 26 of the heart 14.

In some examples, the prosthetic valve delivery apparatus 60 can include an expansion mechanism 65 that is configured to radially expand and deploy the prosthetic heart valve 62 at the implantation site. In some instances, as shown in FIG. 3A, the expansion mechanism 65 can comprise an inflatable balloon that is configured to be inflated to radially expand the prosthetic heart valve 62 within the docking device 52. The inflatable balloon can be coupled to the distal end portion of the delivery shaft 64.

In other examples, the prosthetic heart valve 62 can be self-expanding and can be configured to radially expand on its own upon removable of a sheath or capsule covering the radially compressed prosthetic heart valve 62 on the distal end portion of the delivery shaft 64. In still other examples, the prosthetic heart valve 62 can be mechanically expandable and the prosthetic valve delivery apparatus 60 can include one or more mechanical actuators (for example, the expansion mechanism) configured to radially expand the prosthetic heart valve 62.

As shown in FIG. 3A, the prosthetic heart valve 62 can be mounted around the expansion mechanism 65 (for example, the inflatable balloon) on the distal end portion of the delivery shaft 64, in a radially compressed configuration.

To navigate the distal end portion of the delivery shaft 64 to the implantation site, the user can insert the prosthetic valve delivery apparatus 60 (for example, the delivery shaft 64) into the patient 10 through the guide catheter 30 and over the guidewire 40. The user can continue to advance the prosthetic valve delivery apparatus 60 along the guidewire 40 (for example, through the vasculature 12) until the distal end portion of the delivery shaft 64 reaches the native mitral valve 16, as illustrated in FIG. 3A. More specifically, the user can advance the delivery shaft 64 of the prosthetic valve delivery apparatus 60 by gripping and exerting a force on (for example, pushing) the handle 66. While advancing the delivery shaft 64 through the vasculature 12 and the heart 14, the user can adjust the one or more articulation members 68 of the handle 66 to navigate the various turns, corners, constrictions, and/or other obstacles in the vasculature 12 and heart 14.

The user can advance the delivery shaft 64 along the guidewire 40 until the radially compressed prosthetic heart valve 62 mounted around the distal end portion of the delivery shaft 64 is positioned within the docking device 52 and the native mitral valve 16. In some examples, as shown in FIG. 3A, a distal end of the delivery shaft 64 and a least a portion of the radially compressed prosthetic heart valve 62 can be positioned within the left ventricle 26.

Once the radially compressed prosthetic heart valve 62 is appropriately positioned within the docking device 52 (FIG. 3A), the user can manipulate one or more actuation mechanisms of the handle 66 of the prosthetic valve delivery apparatus 60 to actuate the expansion mechanism 65 (for example, inflate the inflatable balloon), thereby radially expanding the prosthetic heart valve 62 within the docking device 52. In some examples, the user can lock the prosthetic heart valve 62 in its fully expanded position (for example, with a locking mechanism) to prevent the prosthetic heart valve 62 from collapsing.

FIG. 3B shows a fifth stage in the mitral valve replacement procedure where the prosthetic heart valve 62 in its radially expanded configuration and implanted within the docking device 52 in the native mitral valve 16. As shown in FIG. 3B, the prosthetic heart valve 62 can be received and retained within the docking device 52.

As also shown in FIG. 3B, after the prosthetic heart valve 62 has been fully deployed and implanted within the docking device 52 at the native mitral valve 16, the prosthetic valve delivery apparatus 60 (including the delivery shaft 64) can be removed from the patient 10 such that only the guidewire 40 and the guide catheter 30 remain inside the patient 10.

FIG. 4 depicts a sixth stage in the mitral valve replacement procedure, where the guidewire 40 and the guide catheter 30 have been removed from the patient 10. The docking device 52 can be configured to provide a seal between the prosthetic heart valve 62 and the leaflets 24 of the native mitral valve 16 to reduce paravalvular leakage around the prosthetic heart valve 62. Specifically, the docking device 52 can initially constrict the leaflets 24 of the native mitral valve 16. The prosthetic heart valve 62 can then push the leaflets 24 against the docking device 52 as it radially expands within the docking device 52. Thus, the docking device 52 and the prosthetic heart valve 62 can be configured to sandwich the leaflets 24 of the native mitral valve 16 when the prosthetic heart valve 62 is expanded within the docking device 52. In this way, the docking device 52 can provide a seal between the leaflets 24 of the native mitral valve 16 and the prosthetic heart valve 62 to reduce paravalvular leakage around the prosthetic heart valve 62.

In some examples, one or more of the docking device delivery apparatus 50, the prosthetic valve delivery apparatus 60, and/or the guide catheter 30 can comprise one or more fluid ports that are configured to supply flushing fluid to the lumens thereof to prevent and/or reduce the likelihood of blood clot (for example, thrombus) formation. Example fluid ports that can be used to inject flushing fluid into a docking device delivery apparatus are described further below.

Although FIGS. 1-4 specifically depict a mitral valve replacement procedure, it should be appreciated that the same and/or similar procedure may be utilized to replace other heart valves (for example, tricuspid, pulmonary, and/or aortic valves). Further, the same and/or similar delivery apparatuses (for example, docking device delivery apparatus 50, prosthetic valve delivery apparatus 60, guide catheter 30, and/or guidewire 40), docking devices (for example, docking device 52), replacement heart valves (for example, prosthetic heart valve 62), and/or components thereof may be utilized for replacing these other heart valves.

For example, when replacing a native tricuspid valve, the user may also access the right atrium 20 via a femoral vein but may not need to cross the atrial septum 22 into the left atrium 18. Instead, the user may leave the guidewire 40 in the right atrium 20 and perform the same and/or similar docking device implantation process at the tricuspid valve. Specifically, the user may push the docking device 52 out of the delivery shaft 54 around the ventricular side of the tricuspid valve leaflets, release the remaining portion of the docking device 52 from the delivery shaft 54 within the right atrium 20, and then remove the delivery shaft 54 of the docking device delivery apparatus 50 from the patient 10. The user may then advance the guidewire 40 through the tricuspid valve into the right ventricle and perform the same and/or similar prosthetic heart valve implantation process at the tricuspid valve, within the docking device 52. Specifically, the user may advance the delivery shaft 64 of the prosthetic valve delivery apparatus 60 through the patient's vasculature along the guidewire 40 until the prosthetic heart valve 62 is positioned or disposed within the docking device 52 and the tricuspid valve. The user may then expand the prosthetic heart valve 62 within the docking device 52 before removing the prosthetic valve delivery apparatus 60 from the patient 10. In another example, the user may perform the same and/or similar process to replace the aortic valve but may access the aortic valve from the outflow side of the aortic valve via a femoral artery.

Further, although FIGS. 1-4 depict a mitral valve replacement procedure that accesses the native mitral valve 16 from the left atrium 18 via the right atrium 20 and femoral vein, it should be appreciated that the native mitral valve 16 may alternatively be accessed from the left ventricle 26. For example, the user may access the native mitral valve 16 from the left ventricle 26 via the aortic valve by advancing one or more delivery apparatuses through an artery to the aortic valve, and then through the aortic valve into the left ventricle 26.

Additional examples of the docking device delivery apparatus, including its variants, and methods of implanting a docking device and implanting a prosthetic valve within the docking device are described in PCT Patent Application Publication Nos. WO 2020/247907 and WO 2022/087336, and U.S. Patent Publication Nos. US 2018/0318079, US2018/0263764, and US2018/0177594, which are all incorporated by reference herein in their entireties.

### Overview of Docking Devices

Docking devices according to examples of the disclosure can, for example, provide a stable anchoring site, landing zone, or implantation zone at the implant site in which prosthetic valves can be expanded or otherwise implanted. Many of the disclosed docking devices comprise a circular or cylindrically-shaped portion, which can (for example) allow a prosthetic heart valve comprising a circular or cylindrically-shaped valve frame to be expanded or otherwise implanted into native locations with naturally circular cross-sectional profiles and/or in native locations with naturally with non-circular cross sections. In addition to providing an anchoring site for the prosthetic valve, the docking devices can be sized and shaped to cinch or draw the native valve (for example, mitral, tricuspid, etc.) anatomy radially inwards. In this manner, one of the main causes of valve regurgitation (for example, functional mitral regurgitation), specifically enlargement of the heart (for example, enlargement of the left ventricle, etc.) and/or valve annulus, and consequent stretching out of the native valve (for example, mitral, etc.) annulus, can be at least partially offset or counteracted. Some examples of the docking devices further include features which, for example, are shaped and/or modified to better hold a position or shape of the docking device during and/or after expansion of a prosthetic valve therein. By providing such docking devices, replacement valves can be more securely implanted and held at various valve annuluses, including at the mitral valve annulus which does not have a naturally circular cross-section.

In some instances, a docking device can comprise a paravalvular leakage (PVL) guard (also referred to herein as “a guard member”). The PVL guard can, for example, help reduce regurgitation and/or promote tissue ingrowth between the native tissue and the docking device.

The PVL guard can, in some examples, be movable between a delivery configuration and a deployed configuration. When the PVL guard is in the delivery configuration, the PVL guard can extend along and adjacent the coil. When the PVL guard is in the deployed configuration, the PVL guard can form a helical shape rotating about a central longitudinal axis of the coil and at least a segment of the PVL guard can extend radially away from the coil.

In certain examples, the PVL guard can cover or surround a portion of a coil of the docking device. As described more fully below, such PVL guard can move from a radially compressed state to a radially expanded state.

### Exemplary Docking Devices

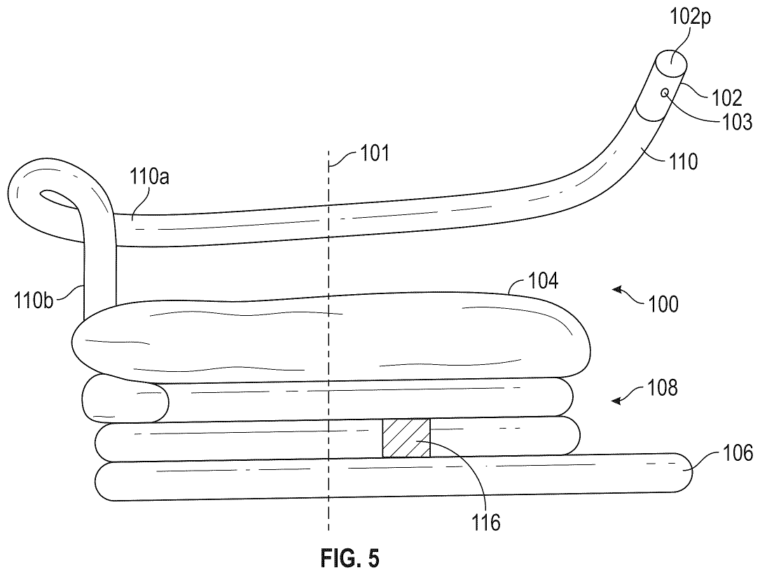

FIG. 5 shows a docking device 100, according to one example. The docking device 100 can, for example, be implanted within a native valve annulus. The docking device 100 can be configured to receive and secure a prosthetic valve (for example, prosthetic heart valve 62), thereby securing the prosthetic valve at the native valve annulus.

The docking device 100 can comprise a coil 102 and a guard member 104 (which can also be referred to as “a PVL guard” or “a sealing member”) covering at least a portion of the coil 102. In certain examples, the coil 102 can include a shape memory material (for example, nickel titanium alloy or “Nitinol”) such that the docking device 100 (and the coil 102) can move from a substantially straight configuration (also referred to as “delivery configuration”) when disposed within a delivery sheath of a delivery apparatus (for example, docking device delivery apparatus 50) to a helical configuration (also referred to as “deployed configuration,” as shown in FIG. 5) after being removed from the delivery sheath.

During delivery of the docking device and after initial deployment of the docking device at the implantation site, the guard member 104 can be retained in a radially compressed state by a dock sleeve of the delivery apparatus. After the docking device 100 is deployed at the implantation site, the dock sleeve can be removed so as to expose the guard member 104, thereby allowing the guard member 104 to move to a radially expanded state.

In some examples, when the docking device 100 is in the deployed configuration and the guard member 104 is in the radially expanded state, the guard member 104 can extend circumferentially relative to a central axis 101 of the docking device 100 from 180 degrees to 400 degrees, or from 210 degrees to 330 degrees, or from 250 degrees to 290 degrees, or from 260 degrees to 280 degrees (for example, 270 degrees) relative to the central axis 101. In other words, the guard member 104 can extend circumferentially from about one half of a revolution (for example, 180 degrees) around the central axis 101 in some examples to more than a full revolution (for example, 400 degrees) around the central axis 101 in other examples, including various ranges in between. As used herein, a range (for example, from 180 degrees to 400 degrees, and between 180 degrees and 400 degrees) includes the endpoints of the range (for example, 180 degrees and 400 degrees).

The coil 102 has a proximal end 102p and a distal end, which also respectively define the proximal and distal ends of the docking device 100. When being disposed within the delivery sheath (for example, during delivery of the docking device into the vasculature of a patient), a body of the coil 102 between the proximal end 102p and distal end can form the generally straight delivery configuration (that is, without any coiled or looped portions, but can be flexed or bent) so as to maintain a small radial profile when moving through a patient's vasculature. After being removed from the delivery sheath and deployed at an implant position, the coil 102 can move from the delivery configuration to the helical deployed configuration and wrap around native tissue adjacent the implant position. For example, when implanting the docking device at the location of a native valve, the coil 102 can be configured to surround native leaflets of the native valve (and the chordae tendineae that connects native leaflets to adjacent papillary muscles, if present).

The docking device 100 can be releasably coupled to a delivery apparatus (for example, docking device delivery apparatus 50). For example, in certain examples, the docking device 100 can be coupled to the delivery apparatus via a release suture that can be configured to be tied to the docking device 100 and cut for removal. In one example, the release suture can be tied to the docking device 100 through an eyelet or eyehole 103 located adjacent the proximal end 102p of the coil. In another example, the release suture can be tied around a circumferential recess that is located adjacent the proximal end 102p of the coil 102.

In some examples, the docking device 100 in the deployed configuration can be configured to fit at the mitral valve position. In other examples, the docking device 100 can also be shaped and/or adapted for implantation at other native valve positions as well, such as at the tricuspid valve. As described herein, the geometry of the docking device 100 can be configured to engage the native anatomy, which can, for example, provide for increased stability and reduction of relative motion between the docking device 100, the prosthetic valve docked therein, and/or the native anatomy. Reduction of such relative motion can, among other things, prevent material degradation of components of the docking device 100 and/or the prosthetic valve docked therein and/or prevent damage or trauma to the native tissue.

As shown in FIG. 5, the coil 102 in the deployed configuration can include a leading turn 106 (or “leading coil”), a central region 108, and a stabilization turn 110 (or “stabilization coil”) around the central axis 101. The central region 108 can possess one or more helical turns having substantially equal inner diameters. The leading turn 106 can extend from a distal end of the central region 108 and has a diameter greater than the diameter of the central region 108 (in one or more configurations). The stabilization turn 110 can extend from a proximal end of the central region 108 and has a diameter greater than the diameter of the central region 108 (in one or more configurations).

In some examples, the central region 108 can include a plurality of helical turns (for example, the docking device 100 can have three helical turns in the central region 108). Some of the helical turns in the central region 108 can be full turns (that is, rotating 360 degrees). In some examples, the most proximal turn and/or the most distal turn can be partial turns (for example, rotating less than 360 degrees, such as 180 degrees, 270 degrees, etc.).

The size of the docking device 100 can be generally selected based on the size of the desired prosthetic valve to be implanted into the patient. In some examples, the central region 108 can be configured to retain a radially expandable prosthetic valve. For example, the inner diameter of the helical turns in the central region 108 can be configured to be smaller than an outer diameter of the prosthetic valve when the prosthetic valve is radially expanded so that additional radial force can act between the central region 108 and the prosthetic valve to hold the prosthetic valve in place. The helical turns in the central region 108 can also be referred to herein as “functional turns.”

The stabilization turn 110 can be configured to help stabilize the docking device 100 in the desired position. For example, the radial dimension of the stabilization turn 110 can be significantly larger than the radial dimension of the coil in the central region 108, so that the stabilization turn 110 can flare or extend sufficiently outwardly so as to abut or push against the walls of the circulatory system, thereby improving the ability of the docking device 100 to stay in its desired position prior to the implantation of the prosthetic valve. In some examples, the diameter of stabilization turn 110 is desirably larger than the native annulus, native valve plane, and/or native chamber for better stabilization. In some examples, the stabilization turn 110 can be a full turn (that is, rotating about 360 degrees). In some examples, the stabilization turn 110 can be a partial turn (for example, rotating between about 180 degrees and about 270 degrees).

In one particularly example, when implanting the docking device 100 at the native mitral valve location, the functional turns in the central region 108 can be disposed substantially in the left ventricle and the stabilization turn 110 can be disposed substantially in the left atrium. The stabilization turn 110 can be configured to provide one or more points or regions of contact between the docking device 100 and the left atrial wall, such as at least three points of contact in the left atrium or complete contact on the left atrial wall. In some examples, the points of contact between the docking device 100 and the left atrial wall can form a plane that is approximately parallel to a plane of the native mitral valve.

In some examples, the stabilization turn 110 can have an atrial portion (covered by the guard member 104 in FIG. 5) in connection with the central region 108, a stabilization portion 110a adjacent to the proximal end 102p of the coil 102, and an ascending portion 110b located between the atrial portion and the stabilization portion 110a. Both the atrial portion and the stabilization portion 110a can be generally parallel to the helical turns in the central region 108, whereas the ascending portion 110b can be oriented to be angular relative to the atrial portion and the stabilization portion 110a. For example, in certain examples, the ascending portion 110 b and the stabilization portion 110 a can form an angle from about 45 degrees to about 90 degrees (inclusive). When implanting the docking device 100 at the native mitral valve location, the atrial portion can be configured to abut the posterior wall of the left atrium and the stabilization portion 110a can be configured to flare out and press against the anterior wall of the left atrium.

As noted above, the leading turn 106 can have a larger radial dimension than the helical turns in the central region 108. The leading turn 106 can help more easily guide the coil 102 around and/or through the chordae tendineae and/or adequately around all native leaflets of the native valve (for example, the native mitral valve, tricuspid valve, etc.). For example, once the leading turn 106 is navigated around the desired native anatomy, the remaining coil (such as the functional turns) of the docking device 100 can also be guided around the same features. In some examples, the leading turn 106 can be a full turn (that is, rotating about 360 degrees). In some examples, the leading turn 106 can be a partial turn (for example, rotating between about 180 degrees and about 270 degrees). When a prosthetic valve is radially expanded within the central region 108 of the coil, the functional turns in the central region 108 can be further radially expanded. As a result, the leading turn 106 can be pulled in the proximal direction and become a part of the functional turn in the central region 108.

As shown in FIG. 8D, at least a portion of the coil 102 can be surrounded by an inner cover 112. The inner cover 112 can have a tubular shape. In some examples, the inner cover 112 can cover an entire length of the coil 102. In some examples, the inner cover 112 covers only selected portion(s) of the coil 102.

In some examples, the inner cover 112 can be coated on and/or bonded on the coil 102. In some examples, the inner cover 112 can be a cushioned, padded-type layer protecting the coil 102. The inner cover 112 can be constructed of various native and/or synthetic materials. In one particularly example, the inner cover 112 can include expanded polytetrafluoroethylene (ePTFE). In some examples, the inner cover 112 is configured to be fixedly attached to the coil 102 (for example, by means of textured surface resistance, suture, glue, thermal bonding, or any other means) so that relative axial movement between the inner cover 112 and the coil 102 is restricted or prohibited.

In some examples, the docking device 100 can also include a retention member 114 (see, for example, FIG. 8D) surrounding at least a portion of the coil 102 (and the inner cover 112) and at least being partially covered by the guard member 104. In some examples, the inner cover 112 can extend through an entire length of the retention member 114. In some examples, at least a portion of the inner cover 112 may not be surrounded by the retention member 114.

In some examples, the retention member 114 can comprise a braided material. In some examples, the retention member 114 can include a woven material. In addition, the retention member 114 can provide a surface area that encourages or promotes tissue ingrowth and/or adherence, and/or reduce trauma to native tissue. For example, in certain instances, the retention member 114 can have a textured outer surface configured to promote tissue ingrowth. In certain instances, the retention member 114 can be impregnated with growth factors to stimulate or promote tissue ingrowth.

In some examples, at least a proximal end portion of the retention member 114 can extend out of (that is, positioned proximal to) a proximal end of the guard member 104. In one particular example, the proximal end of the retention member 114 can be disposed at or adjacent the ascending portion 110b of the coil 102. In some examples, at least a distal end portion of the retention member 114 can extend out of (that is, positioned distal to) a distal end of the guard member 104. In one specific example, a distal end of the retention member 114 can be positioned adjacent the leading turn 106. In some examples, the retention member 114 can cover the functional turns of the coil 102 in the central region 108. Thus, when the docking device 100 is deployed at the native valve and the prosthetic valve is radially expanded within the docking device 100, the retention member 114 at the central region 108 can frictionally engage the prosthetic valve. In some examples, the retention member 114 can be completely covered by the guard member 104.

In some examples, when the docking device 100 is in the deployed configuration, the guard member 104 can be configured to cover a portion (for example, the atrial portion) of the stabilization turn 110 of the coil 102. In certain examples, the guard member 104 can be configured to cover at least a portion of the central region 108 of the coil 102 (for example, a portion of the most proximal turn). In certain examples, the guard member 104 can extend over a majority (or even an entirety) of the functional turns in the central region 108. In one example, when the docking device 100 is deployed at a native atrioventricular valve, the guard member 104 does not extend into the ascending portion 110b to improve the durability of the guard member (for example, to reduce the likelihood of kinking of the guard member).

As described herein, the guard member 104 can radially expand so as to help preventing and/or reducing paravalvular leakage. Specifically, the guard member 104 can be configured to radially expand such that an improved seal is formed closer to and/or against a prosthetic valve deployed within the docking device 100. In some examples, the guard member 104 can be configured to prevent and/or inhibit leakage at the location where the docking device 100 crosses between leaflets of the native valve (for example, at the commissures of the native leaflets). For example, without the guard member 104, the docking device 100 may push the native leaflets apart at the point of crossing the native leaflets and allow for leakage at that point (for example, along the docking device or to its sides). However, the guard member 104 can be configured to expand to cover and/or fill any opening at that point and inhibit leakage along the docking device 100.

In various examples, the guard member 104 can help covering an atrial side of an atrioventricular valve to prevent and/or inhibit blood from leaking through the native leaflets, commissures, and/or around an outside of the prosthetic valve by blocking blood in the atrium from flowing in an atrial to ventricular direction (that is, antegrade blood flow)—other than through the prosthetic valve. Positioning the guard member 104 on the atrial side of the valve can additionally or alternatively help reduce blood in the ventricle from flowing in a ventricular to atrial direction (that is, retrograde blood flow).

In some examples, the guard member 104 can be positioned on a ventricular side of an atrioventricular valve to prevent and/or inhibit blood from leaking through the native leaflets, commissures, and/or around an outside of the prosthetic valve by blocking blood in the ventricle from flowing in a ventricular to atrial direction (that is, retrograde blood flow). Positioning the guard member 104 on the ventricular side of the valve can additionally or alternatively help reduce blood in the atrium from flowing in the atrial direction to ventricular direction (that is, antegrade blood flow)—other than through the prosthetic valve.

In some examples, the docking device 100 can include at least one radiopaque marker configured to provide visual indication about the location of the docking device 100 relative to its surrounding anatomy, and/or the amount of radial expansion of the docking device 100 (for example, when a prosthetic valve is subsequently deployed in the docking device 100) under fluoroscopy. For example, one or more radiopaque markers can be placed on the coil 102. In one particularly example, a radiopaque marker 116 can be disposed at the central region 108 of the coil. In some examples, one or more radiopaque markers can be placed on the inner cover 112, the guard member 104, and/or other components of the docking device. In some examples, the docking device 100 can also have one or more radiopaque markers located distal to the ascending portion 110b of the coil 102 (for example, to ensure the proximal end 104p of the guard member 104 does not extend into the ascending portion 110b).

Further details of the guard member 104 are described in the section “Exemplary PVL Guard” below.

Additional examples of the docking device and its variants, including various examples of the coil, the guard member, the inner cover, and other components of the docking device, are described in PCT Patent Application Publication No. WO/2020/247907, the entirety of which is incorporated by reference herein. Example methods of assembling the guard member are described in PCT Patent Application Publication No. WO 2022/087336 and International Patent Application No. PCT/US 2022/045376, each of which is incorporated by reference herein in its entirety.

### Exemplary Pvl Guards

In any of the examples described herein, the guard member of a docking device can move between a radially compressed state and a radially expanded state. Specifically, the guard member can be biased toward the radially expanded state. As described above, the guard member can be retained in the radially compressed state by a dock sleeve (for example, dock sleeve 105 depicted in FIGS. 8A-8D) of a delivery apparatus, and automatically return to the radially expanded state after the dock sleeve is removed.

In some examples, the guard member can include a shape memory material that is shape set and/or pre-configured to expand to the radially expanded state when unconstrained (for example, when deployed at a native valve location). For example, the guard member can contain a shape memory alloy with super-elastic properties, such as Nitinol. In some examples, the guard member can contain a ternary shape memory alloy with Superelastic properties, such as NiTiX where X can be chromium (Cr), cobalt (Co), zirconium (Zr), hafnium (Hf), etc.

In some examples, the guard member can comprise a metallic material that does not have the shape memory properties. In such circumstances, the guard member can have a biasing mechanism (for example, using springs, etc.) configured to bias the guard member to the radially expanded state. Examples of such metallic material include cobalt-chromium, stainless steel, etc. In one specific example, the guard member can comprise nickel-free austenitic stainless steel in which nickel can be completely replaced by nitrogen. In another specific example, the guard member can comprise cobalt-chromium or cobalt-nickel-chromium-molybdenum alloy with significantly low density of titanium.

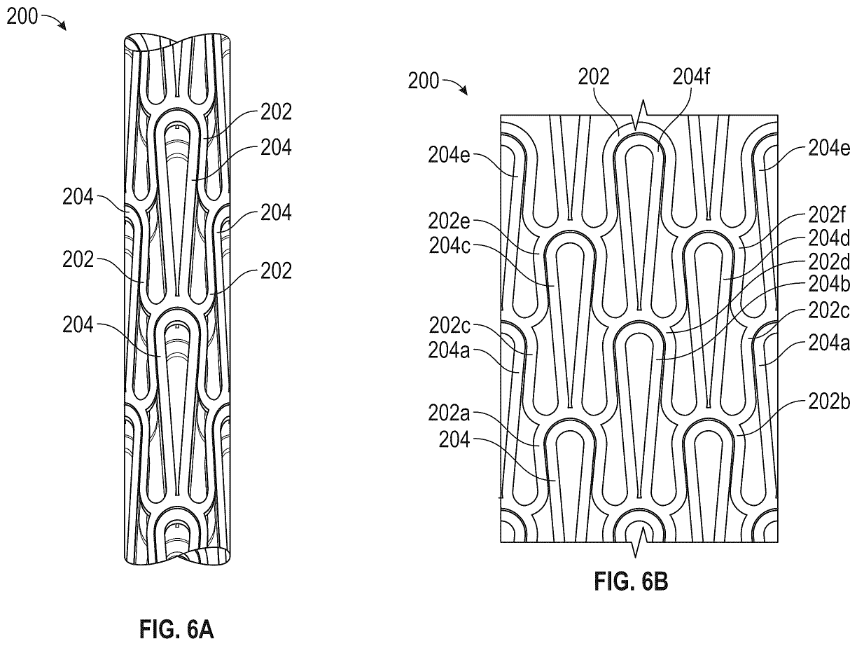

FIG. 6A is a side perspective view of a portion of a guard member 200 in a radially compressed state, according to one example. FIG. 6B is a flattened view of the portion of the guard member of FIG. 6A. The guard member 200 can be one example embodiment of the guard member 104 used in a docking device (for example, the docking device 100), as described above. For example, FIGS. 8A-8D show that the guard member 200 can be mounted over and surround a segment of the coil 102. In FIGS. 8A and 8D, the guard member 200 is retained in a dock sleeve 105 in the radially compressed state. In FIGS. 8B and 8C, the guard member 200 is in the radially expanded state after moving the guard member 200 out of the dock sleeve 105.

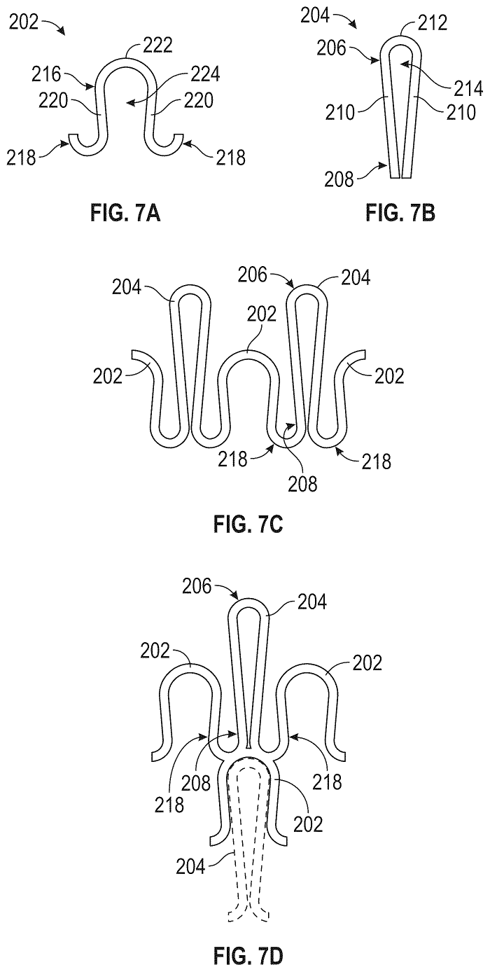

As shown in FIGS. 6A-6B, the guard member 200 includes a plurality of anchor members 202 and a plurality of expandable members 204. Each anchor member 202 is circumferentially positioned between two expandable members 204, and each expandable member 204 is circumferentially positioned between two anchor members 202. The plurality of anchor members 202 and the plurality of expandable members 204 are interconnected to form a unitary piece. An individual anchor member 202 is shown in FIG. 7A, and an individual expandable member 204 is shown in FIG. 7B. Example connections between the anchor members 202 and the expandable members 204 are depicted in FIGS. 7C-7D.

In the examples depicted in FIGS. 6A-6B and 7A-7D, when the guard member 200 is in the radially compressed state, the anchor members 202 are axially shorter than the expandable members 204. In other examples, when the guard member 200 is in the radially compressed state, the anchor members 202 can have about the same axial length as (or even a larger axial length than) the expandable members 204.

As shown in FIGS. 8A-8D, the anchor members 202 are not radially movable relative to the coil 102 when the guard member 200 moves from the radially compressed state to the radially expanded state. In contrast, the expandable members 204 can project radially outwardly from the coil 102 when the guard member 200 moves from the radially compressed state to the radially expanded state. For example, when the guard member 200 is in the radially expanded state, the expandable members 204 can cantilever over respective anchor members 202.

In some examples, the plurality of anchor members 202 are fixedly attached to the coil 102. As a result, the guard member 200 can be stationary (or not axially movable) relative to the coil 102. When the outer surface of the coil 102 is covered by an inner cover 112 (see, for example, FIG. 8D), the plurality of anchor members 202 can directly contact the inner cover 112.

The anchor members 202 can be fixedly attached to the coil 102 via sutures, glue, thermal bonding, or any other means. In some examples, each of the plurality of anchor members 202 can be affixed to the coil 102 (for example, via sutures, glue, thermal bonding, or other means). In some examples, only selected (one, two, or more) anchor members 202 are affixed to the coil 102 (for example, via sutures, glue, thermal bonding, or other means). Because the anchor members 202 are interconnected, so long as one or more anchor members 202 are affixed to the coil 102, the axial positions of other anchor members 202 relative to the coil 102 are also fixed.

As shown in FIG. 7B, each expandable member 204 includes a head portion 206 and a foot portion 208. In some examples, the head portion 206 is wider than the foot portion 208. For example, FIG. 7B shows that the expandable member 204 has a tapered shape with a progressively decreasing width from the head portion 206 to the foot portion 208. In other examples, the expandable member 204 can have a non-tapered shape (for example, the head portion 206 and the foot portion 208 can have about the same width). In still other examples, some of the expandable members can have a tapered shape and other expandable members can have a non-tapered shape.

As shown in FIG. 7B, each expandable member 204 can have two edges 210 (also referred to as “side branches”) and an arc 212 connecting the two edges 210. In the example depicted in FIG. 7B, a hollowed space or cavity 214 is formed between the two edges 210. In other examples, each expandable member 204 can be solid plate (that is, there is no cavity between the two edges 210). In still other examples, some of the expandable members 204 can have cavities 214 and other expandable members 204 can be solid plates.

As shown in FIGS. 6A-6B and 7C-7D, the foot portion 208 of each expandable member can be fixedly connected to two circumferentially adjacent anchor members 202. Because the anchor members 202 are not radially movable relative to the coil 102, the head portion 206 of each expandable member 204 can cantilever over the foot portion 208 of the expandable member 204 when the guard member 200 is in the radially expanded state.

As shown in FIG. 7A, each anchor member 202 comprises a body portion 216 and two side portions 218 located on opposite sides of the body portion 216. The body portion 216 can have two side branches or edges 220 and an arc 222 connecting the two edges 220. A hollowed space or cavity 224 can extend between the two edges 220. Each side portion 218 can have a curved shape and connected to one of the edges 220. As a result, the anchor member 202 can have an omega shape.

As shown in FIGS. 6A-6B and 7C-7D, each side portion 218 of each anchor member 202 can be fixedly connected to the foot portion 208 of an adjacent expandable member 204. Additionally, the foot portion 208 of each expandable member 204 can be fixedly connected to two side portions 218 of two anchor members 202 that are positioned on circumferentially opposite sides of the expandable member 204.

The guard member 200 attached to the coil 102 has two opposite ends. FIGS. 8A-8C depict one end 201 of the guard member 200, which can be a proximal end or a distal end. As shown in FIG. 8A, if the depicted end 201 is the proximal end, the head portion 206 is distal to the foot portion 208 in each expandable member 204 when the guard member 200 is in the radially compressed state. On the other hand, if the depicted end 201 is the distal end, the head portion 206 is proximal to the foot portion 208 in each expandable member 204 when the guard member 200 is in the radially compressed state. In either case, when the guard member 200 is in the radially compressed state, the interconnected expandable members 204 and the anchor members 202 can form a cylindrical mesh extending from the proximal end to the distal end.

As shown in FIGS. 8A-8C, the anchor members 202 can be arranged in a plurality of rows along an axial length of the coil 102. In the example depicted in FIGS. 8A-8C, each row of anchor members has two anchor members 202 that are positioned on opposite sides of the coil 102. In FIGS. 8A-8C, only one anchor member 202 is visible in every other row while the other anchor member 202 in the same row is hidden behind the coil 102. In other examples, each row can have more than two (for example, three, four, or more) anchor members 202 that are equidistantly positioned around a circumference of the coil 102.

As shown in FIGS. 8A-8C, the expandable members 204 can also be arranged in a plurality of rows along the axial length of the coil 102. In the example depicted in FIGS. 8A-8C, each row of expandable members has two expandable members 204 that are positioned on opposite sides of the coil 102. In FIGS. 8A-8C, only one expandable member 204 is visible in every other row while the other expandable member 204 in the same row is hidden behind the coil 102. In other examples, each row can have more than two (for example, three, four, or more) expandable members 204 that are equidistantly positioned around a circumference of the coil 102.

Two adjacent rows of anchor members 202 can be axially offset from one another, regardless the guard member 200 is in the radially compressed state or radially expanded state. For example, FIG. 6B shows a lower row anchor members 202 (formed by two anchor members 202a, 202b), which is axially offset from a middle row of anchor members 202 (formed by two anchor members 202c, 202d), which is axially offset from an upper row of anchor members 202 (formed by two anchor members 202e, 202f).

When the guard member 200 is in the radially compressed state, two adjacent rows of expandable members 204 can axially overlap with one another. For example, FIG. 6B shows a lower row of expandable members 204 (formed by two expandable members 204a, 204b), which axially overlap with a middle row of expandable members 204 (formed by two expandable members 204c, 204d), which axially overlap with an upper row of expandable members 204 (formed by two expandable members 204e, 204f).

In some examples, for each expandable member 204 that is not located at an end (either the proximal end or the distal end) of the guard member 200, the foot portion 208 of the expandable member 204 can be fixedly connected to three adjacent anchor members 202, one of which is axially offset from the expandable member 204. For example, FIG. 6B shows that the foot portion 208 of the expandable member 204f is fixedly connected three anchor members 202e, 202f, and 202d. While two of the anchor members 202e, 202f are positioned on circumferentially opposite sides of the expandable member 204f, the third anchor member 202d has an axial offset relative to the expandable member 204f as the foot portion 208 of the expandable member 204f is connected to the arc 222 of the anchor member 202d.

In some examples, for each expandable member 204 that is not located at an end (either the proximal end or the distal end) of the guard member 200, when the guard member 200 is in the radially compressed state, the head portion 206 of the expandable member 204 can be received within a cavity 224 of an adjacent anchor member 202 that is axially offset from the expandable member 204. For example, FIG. 6B shows that the head portion 206 of the expandable member 204b is received within the cavity 224 of the anchor member 202d, which has an axial offset relative to the expandable member 204b.

In some examples, for each anchor member 202 that is not located at an end (either the proximal end or the distal end) of the guard member 200, the anchor member 202 can be directedly connected to three expandable members 204, one of which is axially offset from the anchor member 202. For example, FIG. 6B shows that the anchor member 202d is directedly connected to three adjacent expandable members 204c, 204d, and 204f.

In some examples, for each anchor member 202 that is not located at an end (either the proximal end or the distal end) of the guard member 200, the anchor member 202 can be directly connected to four other anchor members 202. For example, FIG. 6B shows that the anchor member 202d is directly connected to four neighboring anchor members 202a, 202b, 202e, and 202f.

In some examples, as depicted in FIG. 8C, when the guard member 200 is in the radially expanded state, each expandable member 204 can define a concave surface when viewed from a point on the coil that is axially positioned between the head portion 206 and the foot portion 208 of the expandable member 204.

In some examples, as depicted in FIG. 8B, when the guard member 200 is in the radially expanded state, each expandable member 204 can define a convex surface when viewed from a point on the coil that is axially positioned between the head portion 206 and the foot portion 208 of the expandable member 204.

The curved surface (either convex or concave) of the expandable members 204 depicted in FIGS. 8B and 8C is configured to increase contact surface area between the expandable members 204 and the outer cover 118. Such increase contact surface area can reduce pressure of the expandable members 204 on the outer cover 118. Although the expandable members 204 shown in FIGS. 8B-8C have specific shapes, it should be understood that the exact shape of the expandable members 204 can vary. For example, when the guard member 200 is in the radially expanded state, the foot portion 208 of each expandable member 204 and the coil 102 can have an angle α which can range from about 20° to about 160°, and the head portion 206 and foot portion 208 of each expandable member 204 can have another angle β which can range from about 60° to about 150°.

In some examples, as depicted in FIGS. 8A-8D, a docking device (for example, the docking device 100) can include an outer cover 118 surrounding an outer surface of the guard member 200. In some circumstances, the outer cover 118 can be deemed as a part of the guard member 200, covering the entire structure formed by the interconnected anchor members 202 and expandable members 204.

The outer cover 118 has two opposite ends respectively connected to two opposite ends of the guard member 200. For example, FIGS. 8A-8C depict one end 119 of the outer cover 118 adjacent the end 201 of the guard member 200, which can be a proximal end or a distal end. In some examples, one or both ends of the outer cover 118 can be fixed attached to the coil 102, for example, by sutures or other means.

As described herein, the outer cover 118 can be configured to be so elastic that when the guard member 200 moves from the radially compressed state to the radially expanded state, the outer cover 118 can also radially expand together with the guard member 200, as the expandable members 204 project radially outwardly. The outer cover 118 can be biased toward a radially collapsed configuration in which the outer cover 118 wraps around the guard member 200 in the radially compressed state.

For example, as shown in FIGS. 8B and 8C, when the guard member 200 is in the radially expanded state, the expandable members 204 project radially outwardly from the coil 102, causing the outer cover 118 to radially expand so as to create a cavity, or gap, or void space 111 between the outer cover 118 and the coil 102. As the ends of the outer cover 118 can be fixed attached to the coil 102, each end portion of the outer cover 118 can form a tapered shape while a body portion (between the two ends) of the outer cover 118 can have a generally cylindrical shape.

As shown in FIGS. 8A and 8D, when the guard member 200 is in the radially compressed state, the whole guard member 200 can form a cylindrical mesh wrapping around the coil 102. The elastic outer cover 118 can radially collapse around the guard member 200 so as to leave no gap, cavity, or void space between the outer cover 118 and the coil 102. In the example depicted in FIG. 8D, only the inner cover 112, the retention member 114, and the guard member 200 separate the coil 102 from the outer cover 118.

In certain examples, the outer cover 118 can be configured to be atraumatic to native tissue and/or promote tissue ingrowth into the outer cover 118. For example, the outer cover 118 can have pores to encourage tissue ingrowth. In another example, the outer cover 118 can be impregnated with growth factors to stimulate or promote tissue ingrowth, such as transforming growth factor alpha (TGF-alpha), transforming growth factor beta (TGF-beta), basic fibroblast growth factor (bFGF), vascular epithelial growth factor (VEGF), and combinations thereof. The outer cover 118 can be constructed of any suitable material, including foam, cloth, fabric, and/or polymer, which is flexible to allow for compression and expansion of the outer cover 118. In one example, the outer cover 118 can include a fabric layer constructed from a thermoplastic polymer material, such as polyethylene terephthalate (PET).

In certain examples, the outer cover 118 can be configured to engage with the prosthetic valve deployed within the docking device so as to form a seal and reduce paravalvular leakage between the prosthetic valve and the docking device after the guard member 200 is radially expanded. The outer cover 118 can also be configured to engage with the native tissue (for example, the native annulus and/or native leaflets) to reduce PVL between the docking device and/or the prosthetic valve and the native tissue.

In some examples, the guard member 200 can be made from a cylindrical tube comprising a shape memory material, such as Nitinol. For example, the tube can be laser cut to create a plurality of hollowed spaces. The location, size, and shape of the hollowed spaces are configured so that the uncut portions of the tube can form a cylindrical mesh structure comprising a plurality of anchor members and a plurality of expandable members that are interconnected to form a unitary piece, similar to the example depicted in FIGS. 6A-6B. The expandable members can be shape set so that the expandable members can project radially outwardly or cantilever over respective anchor members when no external force is applied to the plurality of expandable members. The cylindrical mesh structure can be cut to length, resulting in the guard member 200. The guard member 200 can be slid over a portion of a coil (for example, the coil 102). One or more anchor members of the guard member 200 can be fixedly attached to the coil (for example, using suture, glue, thermal bonding, or any other means). An outer cover (for example, the outer cover 118) can be slid over the guard member 200 and tied to the coil at both ends of the outer cover.

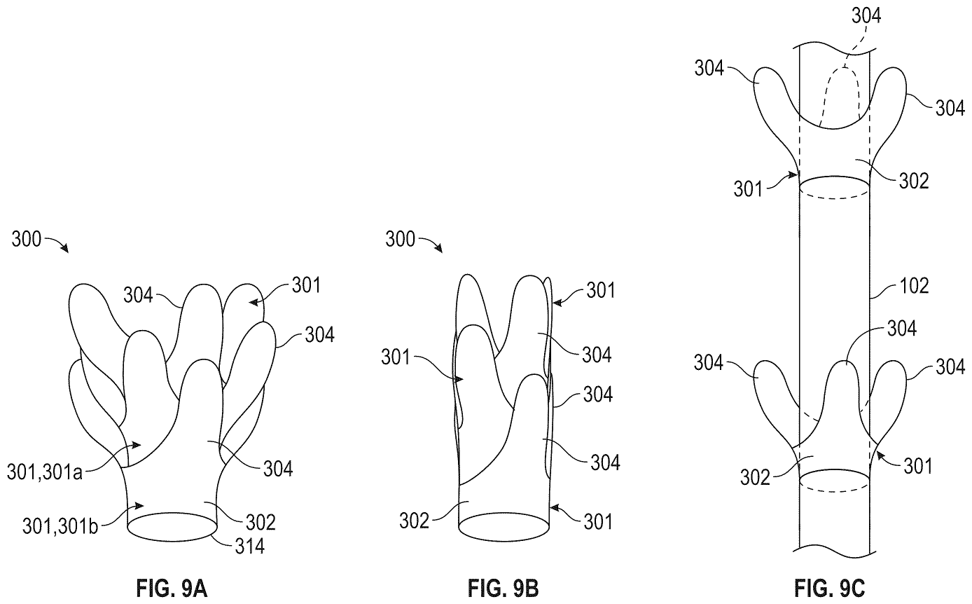

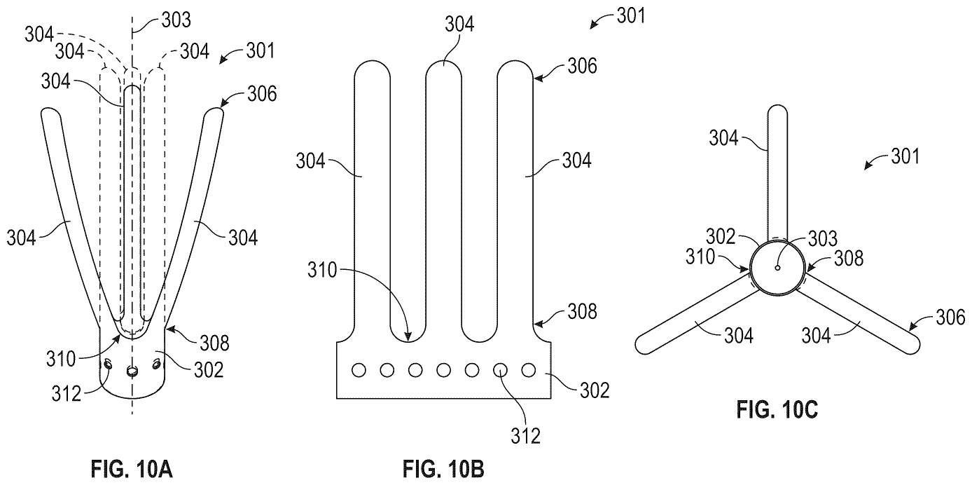

FIGS. 9A and 9B schematically show a portion of a guard member 300, according to another example. FIG. 9A schematically shows the guard member 300 in a radially expanded state and FIG. 9B schematically shows the guard member 300 in a radially compressed state. The guard member 300 includes a plurality of discrete expandable units 301, that is, each expandable unit 301 is a standalone and distinct piece that is not fixedly connected to any other expandable units. A coil (for example, the coil 102) of a docking device (for example, the docking device 100) can extend through the plurality of expandable units 301, thereby stringing the plurality of expandable units 301 together to form a series of stacked expandable units 301 that define the guard member 300. FIG. 9C schematically illustrates sliding two expandable units 301 over a portion of the coil 102. FIG. 10A schematically show a flattened view of one of the expandable units 301. FIG. 10B schematically shows a side view and FIG. 10C schematically shows a top view of the expandable unit 301. The exact shape and/or size of each expandable unit 301 can be the same as or different from the schematic drawings shown in FIGS. 9A-9C and 10A-10C.

As shown in FIGS. 10A-10C, each expandable unit 301 can include one anchor member 302 and two or more expandable members 304 fixedly connected to the anchor member 302. In the depicted example, each expandable unit 301 has three expandable members 304 that are spaced 120 degrees apart from one another in a circumferential direction. In other examples, the number of expandable members 304 in an expandable unit 301 can be two or more than three (for example, four, five, etc.).

The anchor member 302 (which can also be referred to as a “tubular member”) of each expandable unit 301 can have a cylindrical shape configured to encircle or wrap around a segment of the coil 102. In some examples, the diameter of the anchor member 302 can be about the same as the outer diameter of the inner cover 112 surrounding the coil 102.

As shown in FIGS. 10A and 10C, each expandable unit 301 can move between a radially compressed state (indicated by the dashed lines) and a radially expanded state (indicated by the solid lines). In the radially compressed state, all expandable members 304 of the expandable unit 301 extend in the axial direction, parallel to a longitudinal axis 303 of the anchor member 302. In the radially expanded state, all expandable members 304 of the expandable unit 301 project radially outwardly relative to the anchor member 302. For example, the expandable members 304 of each expandable unit 301 can be shape set so that the expandable members 304 can cantilever over the anchor member 302 and project radially outwardly when no external force is applied to the expandable members 304.

As described herein, the guard member 300 is in the radially compressed state when all expandable unit 301 of the guard member 300 are in the radially compressed state, and the guard member 300 is in the radially expanded state when all expandable unit 301 of the guard member 300 are in the radially expanded state.

As shown in FIGS. 10A-10C, each expandable member 304 has a free and movable head portion 306 and a foot portion 308 connected to the anchor member 302. In some examples, the head portions 306 can be wider than the foot portions 308. In some examples, the head portions 306 can be narrower than the foot portions 308. In some examples, the head portions 306 can have about the same width as the foot portions 308.

In some examples, when the expandable unit 301 is in the radially expanded state, each expandable member 304 can define a convex surface when viewed from a point on the anchor member 302 of the expandable unit 301. In some examples, when the expandable unit 301 is in the radially expanded state, each expandable member 304 can define a concave surface when viewed from a point on the anchor member 302 of the expandable unit 301. In some examples, when the expandable unit 301 is in the radially expanded state, each expandable member 304 can define a flat surface.

The plurality of expandable units 301 can be stacked on top of one another and extend from a proximal end to a distal end of the guard member 300. For example, each expandable unit 301 can have a base portion 310 configured to receive an anchor member 302 of another expandable unit 301 that is stacked on top thereof. The base portion 310 can include foot portions 308 of the expandable members 304 and end peripheral regions of the anchor member 302 located between adjacent expandable members 304. When a first expandable unit 301a is stacked over a second expandable unit 301b (for example, around the coil 102), a terminal end 314 of the anchor member 302 of the first expandable unit 301a can abut the base portion 310 of the second expandable unit 301b. As a result, these two adjacent expandable units 301a and 301b are not axially movable relative to one another.

When the guard member 300 is in the radially compressed state, the expandable members 304 of each expandable unit 301 can extend axially along the coil 102 and are axially spaced apart from the anchor member 302 of the expandable unit 301.

In some examples, each two adjacent expandable units 301 can be rotationally movable relative to one another within a limited range of motion. As shown in FIG. 9A, when the first expandable unit 301a is stacked over the second expandable unit 301b, the expandable members 304 of the first expandable unit 301a and the expandable members 304 of the second expandable unit 301b can be arranged in circumferentially alternating positions. Additionally, the expandable members 304 of the first expandable unit 301a can axially overlap with the expandable members 304 of the second expandable unit 301b such that rotational movement of the first expandable unit 301a relative to the second expandable unit 301b is limited by gaps between the expandable members 304 of the first expandable unit 301a and the expandable members 304 of the second expandable unit 301b.

In some examples, when mounting the guard member 300 around the coil 102, two or more expandable units 301 can be fixedly attached to the coil 102. In some examples, an expandable unit 301 can be affixed to the coil 102 by one or more sutures. For example, the anchor member 302 of an expandable unit 301 can comprise a plurality of apertures 312 through which a suture can extend through to affix the expandable unit 301 to the coil 102. In some examples, the anchor member 302 of an expandable unit 301 can be affixed to the coil 102 by other means, such as using glue, thermal bonding, interference fit, etc.

In some examples, among the plurality of expandable units 301 forming the guard member 300, a proximal expandable unit located at the proximal end of the guard member and a distal expandable unit located at the distal end of the guard member can be fixed attached to the coil 102. As a result, both the proximal and distal ends of the guard member 300 are stationary relative to the coil 102. In some examples, some of the expandable units 301 located between the proximal and distal expandable units can also be fixed attached to the coil 102 whereas other expandable units 301 located between the proximal and distal expandable units are not affixed to the coil 102.

As a result, the guard member 300 can comprise a series of loosely connected expandable units 301. Except for those expandable units 301 fixedly attached to the coil 102 which can neither rotate nor move axially relative to the coil 102, the other expandable units 301 can rotate relative to the coil 102 within a limited range but cannot move axially relative to the coil 102. Such loosely connected expandable units 301 provide improved structural and shape flexibility for the guard member 300. For example, when a docking device (for example, the docking device 100) comprising the guard member 300 is deployed as a native annulus, the flexible guard member 300 can more easily conform to the geometry of surrounding native anatomy and/or the prosthetic valve deployed within the docking device, thereby providing improved paravalvular sealing and/or reducing the risk of damage to surrounding native tissues and/or the prosthetic valve.

In some examples, a docking device (for example, the docking device 100) having the guard member 300 can also include an outer cover similar to the outer cover 118 described above. In some circumstances, the outer cover can be deemed as a part of the guard member 300, covering all expandable units 301 of the guard member 300. Similarly, the outer cover can be configured to be so elastic that when the guard member 300 moves from the radially compressed state to the radially expanded state, the outer cover can also radially expand together with the guard member 300, as the expandable members 304 project radially outwardly. The outer cover can be biased toward a radially collapsed configuration in which the outer cover wraps around the guard member 300 in the radially compressed state.

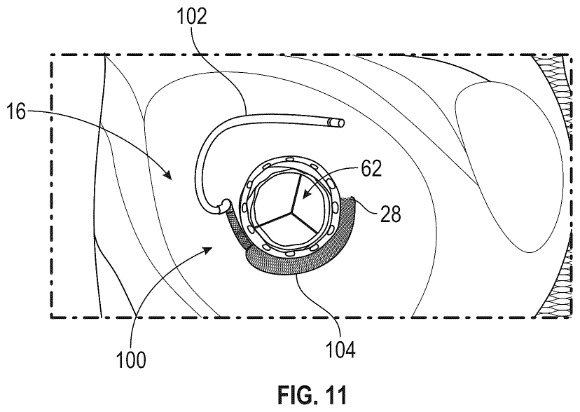

FIG. 11 shows the docking device 100 having the guard member 104 deployed at the annulus of a native mitral valve 16 and a prosthetic valve 62 is radially expanded within the docking device 100. The guard member 104 can be the guard member 200 or 300 described above. In this example, the implantation site is a mitral annulus that separates the left atrium from the left ventricle (the view is from the left atrial side and the left ventricle is behind the mitral annular plane). The guard member 104 can extend through the mitral annulus at a location adjacent to a medial commissure 28 of the native mitral valve so that leakage at that location can be prevented or reduced.

### Exemplary Prosthetic Valves

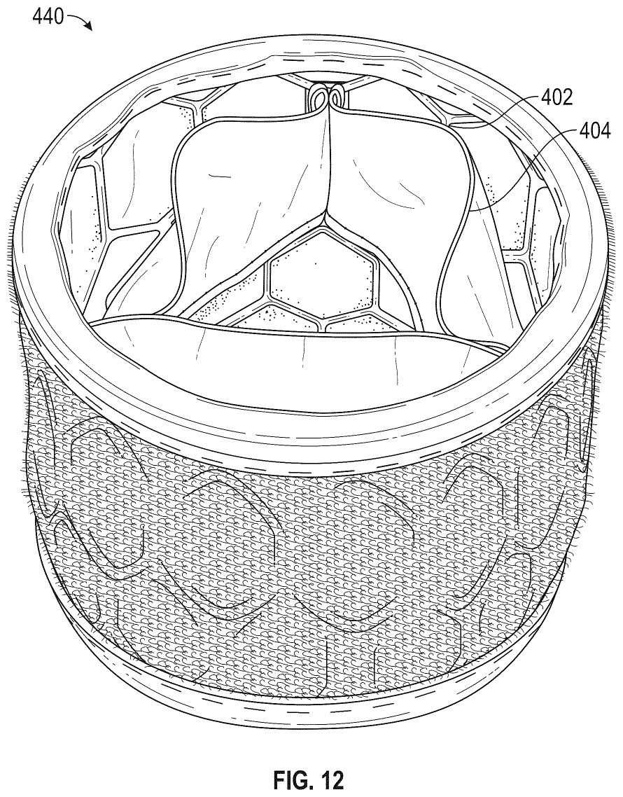

FIG. 12 shows a prosthetic heart valve 400, which can be one specific example of the prosthetic valve 62 described above. As shown, the heart valve 400 comprises a frame, or stent, 402 and a leaflet structure 404 supported by the frame. In some examples, the prosthetic heart valve 400 is adapted to be implanted in the native aortic valve and can be implanted in the body using, for example, the prosthetic valve delivery apparatus 60 described above. The prosthetic valve 400 can also be implanted within the body using any of the other delivery apparatuses described herein.

In some examples, the frame 402 comprises a plastically expandable material, which can be metal alloys, polymers, or combinations thereof. Example metal alloys can comprise one or more of the following: nickel, cobalt, chromium, molybdenum, titanium, or other biocompatible metal. In some examples, the frame 402 can comprise stainless steel. In some examples, the frame 402 can comprise cobalt-chromium. In some examples, the frame 402 can comprise nickel-cobalt-chromium. In some examples, the frame 402 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

In some examples, the prosthetic valve 62 or 400 can be a self-expandable prosthetic valve with a frame made from a self-expanding material, such as Nitinol. When the prosthetic valve is a self-expanding valve, the balloon of the delivery apparatus can be replaced with a sheath or similar restraining device that retains the prosthetic valve in a radially compressed state for delivery through the body. When the prosthetic valve is at the implantation location, the prosthetic valve can be released from the sheath, and therefore allowed to expand to its functional size. It should be noted that any of the delivery apparatuses disclosed herein can be adapted for use with a self-expanding valve.

Additional details regarding the prosthetic valves described herein and various valve components are described U.S. Pat. No. 11,185,406, which is incorporated herein by reference. Additional example prosthetic valves are described in International Patent Application Publication No. WO 2018/222799, U.S. Pat. No. 9,155,619, and U.S. Patent Publication No. 2018/0028310, all of which are incorporated herein by reference in their entireties.

### Sterilization

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A docking device for securing a prosthetic valve at a native valve, the docking device comprising: a coil comprising a plurality of helical turns when deployed at the native valve; and a guard member attached to the coil and is movable between a radially compressed state and a radially expanded state, wherein the guard member comprises a plurality of anchor members and a plurality of expandable members, wherein the anchor members are not radially movable relative to the coil when the guard member moves from the radially compressed state to the radially expanded state, wherein the expandable members project radially outwardly from the coil when the guard member moves from the radially compressed state to the radially expanded state.

Example 2. The docking device of any example herein, particularly example 1, wherein the plurality of anchor members and the plurality of expandable members are interconnected to form a unitary piece.

Example 3. The docking device of any example herein, particularly example 2, wherein each anchor member is circumferentially positioned between two expandable members, and each expandable member is circumferentially positioned between two anchor members.

Example 4. The docking device of any example herein, particularly example 3, wherein each expandable member comprises a head portion and a foot portion, wherein the foot portion of each expandable member is fixedly connected to two circumferentially adjacent anchor members.

Example 5. The docking device of any example herein, particularly example 4, wherein the head portion of each expandable member cantilevers over the foot portion of the expandable member when the guard member is in the radially expanded state.

Example 6. The docking device of any example herein, particularly example 5, wherein the guard member comprises a proximal end and a distal end, wherein when the guard member is in the radially compressed state, the plurality of expandable members and the plurality of anchor members form a cylindrical mesh extending from the proximal end to the distal end.

Example 7. The docking device of any example herein, particularly example 6, wherein for each expandable member that is not located at the proximal end or the distal end, the foot portion of the expandable member is fixedly connected to three adjacent anchor members, one of which is axially offset from the expandable member.

Example 8. The docking device of any example herein, particularly any one of examples 6-7, wherein for each expandable member that is not located at the proximal end or the distal end, when the guard member is in the radially compressed state, the head portion of the expandable member is received within a hollowed space of an adjacent anchor member that is axially offset from the expandable member.

Example 9. The docking device of any example herein, particularly any one of examples 6-8, wherein each anchor member that is not located at the proximal end or the distal end is directly connected to three expandable members, one of which is axially offset from the anchor member.

Example 10. The docking device of any example herein, particularly any one of examples 6-8, wherein each anchor member that is not located at the proximal end or the distal end is directly connected to four other anchor members.

Example 11. The docking device of any example herein, particularly any one of examples 4-10, wherein the head portion is wider than the foot portion.

Example 12. The docking device of any example herein, particularly any one of examples 4-11, wherein when the guard member is in the radially expanded state, each expandable member defines a concave surface when viewed from a point on the coil that is axially positioned between the head portion and the foot portion of the expandable member.

Example 13. The docking device of any example herein, particularly any one of examples 4-11, wherein when the guard member is in the radially expanded state, each expandable member defines a convex surface when viewed from a point on the coil that is axially positioned between the head portion and the foot portion of the expandable member.

Example 14. The docking device of any example herein, particularly any one of examples 2-13, wherein each expandable member comprises two side branches and an arc connecting the two side branches.

Example 15. The docking device of any example herein, particularly any one of examples 2-13, wherein each expandable member is a solid plate.

Example 16. The docking device of any example herein, particularly any one of examples 2-15, wherein each anchor member has an omega shape.

Example 17. The docking device of any example herein, particularly any one of examples 2-16, wherein the plurality of anchor members are arranged in a plurality of first rows along an axial length of the coil.

Example 18. The docking device of any example herein, particularly example 17, wherein each first row comprises two anchor members that are on opposite sides of the coil.

Example 19. The docking device of any example herein, particularly any one of examples 17-18, wherein two adjacent first rows of anchor members are axially offset from one another.

Example 20. The docking device of any example herein, particularly any one of examples 17-19, wherein the plurality of expandable members are arranged in a plurality of second rows along the axial length of the coil.

Example 21. The docking device of any example herein, particularly example 20, wherein each second row comprises two expandable members that are on opposite sides of the coil.

Example 22. The docking device of any example herein, particularly any one of examples 20-21, wherein two adjacent second rows of expandable members axially overlap with one another when the guard member is in the radially compressed state.

Example 23. The docking device of any example herein, particularly any one of examples 2-22, wherein when the guard member is in the radially compressed state, the plurality of anchor members are axially shorter than the plurality of expandable members.

Example 24. The docking device of any example herein, particularly example 1, wherein the guard member comprises a plurality of discrete expandable units, each expandable unit comprising one anchor member and two or more expandable members fixedly connected to the anchor member.

Example 25. The docking device of any example herein, particularly example 24, wherein when the guard member is in the radially expanded state, the two or more expandable members cantilever over the anchor member in each expandable unit.

Example 26. The docking device of any example herein, particularly any one of examples 24-25, wherein when the guard member is in the radially compressed state, the two or more expandable members of each expandable unit extend axially along the coil and are axially spaced apart from the anchor member of the expandable unit.

Example 27. The docking device of any example herein, particularly any one of examples 24-26, wherein the anchor member in each expandable unit has a cylindrical shape and is configured to encircle a segment of the coil.

Example 28. The docking device of any example herein, particularly any one of examples 24-27, wherein when the guard member is in the radially expanded state, each expandable member of each expandable unit defines a convex surface when viewed from a point on the anchor member of the expandable unit.

Example 29. The docking device of any example herein, particularly any one of examples 24-27, wherein when the guard member is in the radially expanded state, each expandable member of each expandable unit defines a concave surface when viewed from a point on the anchor member of the expandable unit.

Example 30. The docking device of any example herein, particularly any one of examples 24-29, wherein the plurality of expandable units are stacked on one another and extend from a proximal end of the guard member to a distal end of the guard member.

Example 31. The docking device of any example herein, particularly example 30, wherein each expandable unit comprises a base portion configured to receive an anchor member of another expandable unit that is stacked on top thereof.

Example 32. The docking device of any example herein, particularly any one of examples 30-31, wherein each two adjacent expandable units are not axially movable relative to one another.

Example 33. The docking device of any example herein, particularly any one of examples 30-32, wherein each two adjacent expandable units are rotationally movable relative to one another.

Example 34. The docking device of any example herein, particularly example 33, wherein when a first expandable unit is stacked on a second expandable unit, the expandable members of the first expandable unit and the expandable members of the second expandable unit are arranged in circumferentially alternating positions.

Example 35. The docking device of any example herein, particularly example 34, wherein the expandable members of the first expandable unit axially overlap with the expandable members of the second expandable unit such that rotational movement of the first expandable unit relative to the second expandable unit is limited by gaps between the expandable members of the first expandable unit and the expandable members of the second expandable unit.

Example 36. The docking device of any example herein, particularly any one of examples 30-35, wherein two or more expandable units are fixedly attached to the coil.

Example 37. The docking device of any example herein, particularly example 36, wherein the two or more expandable units fixedly attached to the coil comprise a proximal expandable unit located at the proximal end of the guard member and a distal expandable unit located at the distal end of the guard member.

Example 38. The docking device of any example herein, particularly any one of examples 36-37, wherein the anchor member of each expandable unit that is fixedly attached to the coil comprises a plurality of apertures through which a suture can extend through to affix the expandable unit to the coil.

Example 39. The docking device of any example herein, particularly any one of examples 24-38, wherein each expandable unit comprises three expandable members that are circumferentially equidistant from each other.

Example 40. The docking device of any example herein, particularly any one of examples 1-39, wherein the guard member is not axially movable relative to the coil.

Example 41. The docking device of any example herein, particularly any one of examples 1-40, wherein the guard member comprise a shape memory alloy.

Example 42. The docking device of any example herein, particularly any one of examples 1-41, further comprising an outer cover surrounding an outer surface of the guard member.

Example 43. The docking device of any example herein, particularly any one of examples 1-42, further comprising an inner cover fixedly attached to an outer surface of the coil, wherein the plurality of anchor members directly contact the inner cover.

Example 44. A docking device for securing a prosthetic valve at a native valve, the docking device comprising: a coil comprising a plurality of helical turns when deployed at the native valve; and a guard member attached to the coil and is movable between a radially compressed state and a radially expanded state, wherein the guard member comprises a plurality of anchor members fixedly attached to the coil and a plurality of expandable members that can cantilever over respective anchor members, wherein the guard member is axially stationary relative to the coil.

Example 45. The docking device of any example herein, particularly example 44, wherein the expandable members project radially outwardly from the coil when the guard member moves from the radially compressed state to the radially expanded state.

Example 46. The docking device of any example herein, particularly any one of examples 42-43, wherein the plurality of expandable members comprise Nitinol.

Example 47. A docking device for securing a prosthetic valve at a native valve, the docking device comprising: a coil comprising a plurality of helical turns when deployed at the native valve; and a guard member attached to the coil and is movable between a radially compressed state and a radially expanded state, wherein the guard member comprises a plurality of anchor members fixedly attached to the coil and a plurality of expandable members that can cantilever over respective anchor members, wherein the plurality of anchor members and the plurality of expandable members are interconnected to form a unitary piece.

Example 48. A docking device for securing a prosthetic valve at a native valve, the docking device comprising: a coil comprising a plurality of helical turns when deployed at the native valve; and a guard member attached to the coil and is movable between a radially compressed state and a radially expanded state, wherein the guard member comprises a plurality of discrete expandable units, each expandable unit comprising one anchor member and two or more expandable members that are fixedly connected to and can cantilever over the anchor member.

Example 49. A method of making a docking device for securing a prosthetic valve at a native valve, the method comprising: receiving a tube comprising a shape memory material; transforming the tube into a cylindrical mesh structure comprising a plurality of anchor members and a plurality of expandable members that are interconnected to form a unitary piece; and shape setting the plurality of expandable members so that the plurality of expandable members cantilever over respective anchor members and project radially outwardly when no external force is applied to the plurality of expandable members.

Example 50. The method of any example herein, particularly example 49, wherein the transforming comprises laser cutting the tube to create a plurality of hollowed spaces between the plurality of anchor members and the plurality of expandable members.

Example 51. The method of any example herein, particularly any one of examples 49-50, further comprising sliding the cylindrical mesh structure over a portion of a coil, wherein the coil is configured to move from a substantially straight configuration to a helical configuration when deployed at the native valve.

Example 52. The method of any example herein, particularly example 51, further comprising fixedly attaching at least some of the plurality of anchor members to the coil.

Example 53. The method of any example herein, particularly any one of examples 49-52, further comprising covering the cylindrical mesh structure with an outer cover.

Example 54. A method of making a docking device for securing a prosthetic valve at a native valve, the method comprising: receiving a plurality of discrete expandable units, each expandable unit comprising one tubular member and two or more expandable members that are fixedly connected to the tubular member; and sliding the plurality of expandable units over a portion of a coil so that the plurality of expandable units are stacked on one another, wherein the coil is configured to move from a substantially straight configuration to a helical configuration when deployed at the native valve.

Example 55. The method of any example herein, particularly example 54, further comprising affixing tubular members of two or more expandable units to the coil.

Example 56. The method of any example herein, particularly any one of examples 54-55, further comprising shape setting the two or more expandable members of each expandable unit so that the two or more expandable members cantilever over the tubular members and project radially outwardly when no external force is applied to the two or more expandable members.

Example 57. A method comprising: deploying a docking device of any example herein, particularly any one of examples 1-48 at an annulus of a native valve; and deploying a prosthetic valve within the docking device.

Example 58. A method comprising sterilizing the docking device of any example herein, particularly any one of examples 1-48.

Example 59. A method of treating a heart on a simulation, the method comprising:
deploying a docking device at a target location; and deploying a prosthetic valve within the docking device; wherein the docking device is according to any one of examples 1-48.

The features described herein with regard to any example can be combined with other features described in any one or more of the other examples, unless otherwise stated. For example, any one or more of the features of one docking device can be combined with any one or more features of another docking device.

In view of the many possible examples to which the principles of the disclosed technology may be applied, it should be recognized that the illustrated examples are only preferred examples of the technology and should not be taken as limiting the scope of the disclosure. Rather, the scope of the claimed subject matter is defined by the following claims and their equivalents.

What is claimed is:

1. A docking device for securing a prosthetic valve at a native valve, the docking device comprising:
a coil comprising a plurality of helical turns when deployed at the native valve; and
a guard member attached to the coil and is movable between a radially compressed state and a radially expanded state,
wherein the guard member comprises a plurality of anchor members and a plurality of expandable members,
wherein the anchor members are not radially movable relative to the coil when the guard member moves from the radially compressed state to the radially expanded state,
wherein the expandable members project radially outwardly from the coil when the guard member moves from the radially compressed state to the radially expanded state.

2. The docking device of claim 1, wherein the plurality of anchor members and the plurality of expandable members are interconnected to form a unitary piece.

3. The docking device of claim 2, wherein each anchor member is circumferentially positioned between two expandable members, and each expandable member is circumferentially positioned between two anchor members.

4. The docking device of claim 3, wherein each expandable member comprises a head portion and a foot portion, wherein the foot portion of each expandable member is fixedly connected to two circumferentially adjacent anchor members.

5. The docking device of claim 4, wherein the head portion of each expandable member cantilevers over the foot portion of the expandable member when the guard member is in the radially expanded state.

6. The docking device of claim 5, wherein the guard member comprises a proximal end and a distal end, wherein when the guard member is in the radially compressed state, the plurality of expandable members and the plurality of anchor members form a cylindrical mesh extending from the proximal end to the distal end.

7. The docking device of claim 6, wherein for each expandable member that is not located at the proximal end or the distal end, the foot portion of the expandable member is fixedly connected to three adjacent anchor members, one of which is axially offset from the expandable member.

8. The docking device of claim 6, wherein for each expandable member that is not located at the proximal end or the distal end, when the guard member is in the radially compressed state, the head portion of the expandable member is received within a hollowed space of an adjacent anchor member that is axially offset from the expandable member.

9. The docking device of claim 6, wherein each anchor member that is not located at the proximal end or the distal end is directly connected to three expandable members, one of which is axially offset from the anchor member.

10. The docking device of claim 6, wherein each anchor member that is not located at the proximal end or the distal end is directly connected to four other anchor members.

11. The docking device of claim 4, wherein the head portion is wider than the foot portion.

12. The docking device of claim 4, wherein when the guard member is in the radially expanded state, each expandable member defines a concave surface when viewed from a point on the coil that is axially positioned between the head portion and the foot portion of the expandable member.

13. The docking device of claim 4, wherein when the guard member is in the radially expanded state, each expandable member defines a convex surface when viewed from a point on the coil that is axially positioned between the head portion and the foot portion of the expandable member.

14. The docking device of claim 2, wherein each expandable member comprises two side branches and an arc connecting the two side branches.

15. The docking device of claim 2, wherein each expandable member is a solid plate.

16. The docking device of claim 2, wherein each anchor member has an omega shape.

17. A docking device for securing a prosthetic valve at a native valve, the docking device comprising:
a coil comprising a plurality of helical turns when deployed at the native valve; and
a guard member attached to the coil and is movable between a radially compressed state and a radially expanded state,
wherein the guard member comprises a plurality of anchor members fixedly attached to the coil and a plurality of expandable members that can cantilever over respective anchor members,
wherein the guard member is axially stationary relative to the coil.

18. The docking device of claim 17, wherein the expandable members project radially outwardly from the coil when the guard member moves from the radially compressed state to the radially expanded state.

19. The docking device of claim 17, wherein the plurality of expandable members comprises Nitinol.

20. A method of making a docking device for securing a prosthetic valve at a native valve, the method comprising:
receiving a tube comprising a shape memory material;
transforming the tube into a cylindrical mesh structure comprising a plurality of anchor members and a plurality of expandable members that are interconnected to form a unitary piece; and
shape setting the plurality of expandable members so that the plurality of expandable members cantilever over respective anchor members and project radially outwardly when no external force is applied to the plurality of expandable members.
